SEQUENCE LISTING (38 sequences)

SEQ ID NO: 1 - DNA, 180 bp, Bacillus anthracis
cgaactcattgaactaactgataagagcatgaatacattgattaaaatgtccagtgtaccagaaaatagaattttagatggcggaaaagctaatatagtaaagtaataattttatttatgaatttacttctaaaaagcagatagaaataaaattctagttttagacaggagattcgatat

SEQ ID NO: 2 - DNA, 23 bp, Artificial Sequence
  Oligonucleotide primer
tggcggaaaagctaatatagtaa

SEQ ID NO: 3 - DNA, 23 bp, Artificial Sequence
  Oligonucleotide primer
gattaaaatgtccagtgtaccag

SEQ ID NO: 4 - DNA, 29 bp, Artificial Sequence
  Oligonucleotide primer
gtggcggaaaagctaatatagtaagccac

SEQ ID NO: 5 - DNA, 5 bp, Artificial Sequence
  Oligonucleotide primer
gtggc

SEQ ID NO: 6 - DNA, 5 bp, Artificial Sequence
  Oligonucleotide primer
gccac

SEQ ID NO: 7 - DNA, 30 bp, Artificial Sequence
  Oligonucleotide primer
gtggcggaaaagctaatatagtaacgccac

SEQ ID NO: 8 - DNA, 6 bp, Artificial Sequence
  Oligonucleotide primer
gtggcg

SEQ ID NO: 9 - DNA, 6 bp, Artificial Sequence
  Oligonucleotide primer
cgccac

SEQ ID NO: 10 - DNA, 31 bp, Artificial Sequence
  Oligonucleotide primer
gtggcggaaaagctaatatagtaaccgccac

SEQ ID NO: 11 - DNA, 7 bp, Artificial Sequence
  Oligonucleotide primer
gtggcgg

SEQ ID NO: 12 - DNA, 7 bp, Artificial Sequence
  Oligonucleotide primer
ccgccac

SEQ ID NO: 13 - DNA, 33 bp, Artificial Sequence
  Oligonucleotide primer
ggtggcggaaaagctaatatagtaaccgccacc

SEQ ID NO: 14 - DNA, 8 bp, Artificial Sequence
  Oligonucleotide primer
ggtggcgg

SEQ ID NO: 15 - DNA, 8 bp, Artificial Sequence
  Oligonucleotide primer
ccgccacc

SEQ ID NO: 16 - DNA, 35 bp, Artificial Sequence
  Oligonucleotide primer
gggtggcggaaaagctaatatagtaaccgccaccc

SEQ ID NO: 17 - DNA, 9 bp, Artificial Sequence
  Oligonucleotide primer
gggtggcgg

SEQ ID NO: 18 - DNA, 9 bp, Artificial Sequence
  Oligonucleotide primer
ccgccaccc

SEQ ID NO: 19 - DNA, 19 bp, Artificial Sequence
  Oligonucleotide primer
aactaactgataagagcat

SEQ ID NO: 20 - DNA, 17 bp, Bacillus anthracis
tatcgaatctcctgtct

SEQ ID NO: 21 - DNA, 80 bp, Artificial Sequence
  Oligonucleotide primer
gaatacattgattaaaatgtccagtgtaccagaaaatagaattttagatggcggaaaagctaatatagtaaagtaataat

SEQ ID NO: 22 - DNA, 80 bp, Artificial Sequence
  Oligonucleotide primer
gaatacattgattaaaatatccagtgtaccagaaaatagaattttagatggcggaaaagctaatatagtaaagtaataat

SEQ ID NO: 23 - DNA, 80 bp, Artificial Sequence
  Oligonucleotide primer
gaatacattgattaaaatgtccagtgtaccagaaaatagaattttagatggcggaaaagataatatagtaaagtaataat

SEQ ID NO: 24 - DNA, 80 bp, Artificial Sequence
  Oligonucleotide primer
gaatacattgattaaaatatccagtgtaccagaaaatagaattttagatggcggaaaagataatatagtaaagtaataat

SEQ ID NO: 25 - DNA, 19 bp, Artificial Sequence
  Oligonucleotide primer
ataaagggaaagtataccg

SEQ ID NO: 26 - DNA, 30 bp, Artificial Sequence
  Oligonucleotide primer
cttctacgcatgaccatattcgcgtagaag

SEQ ID NO: 27 - DNA, 9 bp, Artificial Sequence
  Oligonucleotide primer
cttctacgc

SEQ ID NO: 28 - DNA, 9 bp, Artificial Sequence
  Oligonucleotide primer
gcgtagaag

SEQ ID NO: 29 - DNA, 24 bp, Artificial Sequence
  Oligonucleotide primer
gggaacaaatgatgatgatttcgt

SEQ ID NO: 30 - DNA, 25 bp, Artificial Sequence
  Oligonucleotide primer
actctgggatttcatatcctttcgt

SEQ ID NO: 31 - DNA, 17 bp, Artificial Sequence
  Oligonucleotide primer
gagtattcgtctggggg

SEQ ID NO: 32 - DNA, 30 bp, Artificial Sequence
  Oligonucleotide primer
ccccgaggttcaggtgagcacgctcgggga

SEQ ID NO: 33 - DNA, 7 bp, Artificial Sequence
  Oligonucleotide primer
ccccgag

SEQ ID NO: 34 - DNA, 7 bp, Artificial Sequence
  Oligonucleotide primer
ctcgggg

SEQ ID NO: 35 - DNA, 18 bp, Artificial Sequence
  Oligonucleotide primer
gcaggaaatgcgcaatgc

SEQ ID NO: 36 - DNA, 18 bp, Artificial Sequence
  Oligonucleotide primer
gggcggatccccacttta

SEQ ID NO: 37 - DNA, 21 bp, Artificial Sequence
  Oligonucleotide primer
cttctacgcatgaccatattc

SEQ ID NO: 38 - DNA, 19 bp, Artificial Sequence
  Oligonucleotide primer
cgaggttcaggtgagcacg

(19) United States
(12) Patent Application Publication
(10) Pub. No.: US 2009/0305264 A1
(43) Pub. Date: Dec. 10, 2009

(54) COOPERATIVE PROBES AND METHODS OF USING THEM

(76) Inventors: Jason A.A. West, Castro Valley, CA (US); Brent C. Satterfield, Mesa, AZ (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/296,000

(22) PCT Filed: Mar. 2, 2007
(86) PCT No.: PCT/US2007/063229

§ 371 Date: Jul. 31, 2009

Related U.S. Application Data
(60) Provisional application No. 60/789,267, filed on Apr. 4, 2006.
(60) Provisional application No. 60/801,543, filed on May 17, 2006.
(60) Provisional application No. 60/850,958, filed on Oct. 10, 2006.

(51) Int. Cl. C12Q 1/68; C07H 21/04

(52) U.S. Cl. 435/6; 536/24.3

(57) ABSTRACT

The present invention provides inter alia, cooperative probe assays for analyzing and identifying biological substances.

### CROSS REFERENCE TO RELATED APPLICATION

This application claims the benefits of U.S. Provisional Application No. 60/789,267 filed Apr. 4, 2006, U.S. Provisional Application No. 60/801,543 filed May 17, 2006 and U.S. Provisional Application No. 60/850,958 filed Oct. 10, 2006, each of which is herein incorporated by reference in its entirety.

### FIELD

The present invention is related to the field of molecular recognition in biosensors. In particular, the present invention is related to assays and methods for analyzing and identifying biological substances. The present invention is also related to molecules having structures that facilitate cooperativity for enhanced performance.

### BACKGROUND

Field-deployable biosensors require more rapid and sensitive, single-step identification methods. However, efforts to enhance assay rapidity, sensitivity and simplicity can result in an increase in false positives and false negatives. Such false positives and negatives can have immense impact in biosensing for medical and biowarfare applications. Even rare occurrences can have disastrous consequences. Understanding and designing assay formats for the specificity-sensitivity tradeoff is absolutely essential to developing field-deployable biosensors exhibiting few to essentially no false positives and negatives.

Molecular beacons are a class of fluorescence-quenched nucleic acid probes that can be used to enhance the performance of rapid, single-step sensors (Drake and Tan (2004) Appl. Spectrosc. 58(9):269A-280A; Marras et al. (2006) Clin. Chim. Acta 363(1-2):48-60). A fluorescent label is attached to one end of a polynucleotide and a quencher is attached to the other. Complementary base-pairs near the label and quencher cause a hairpin-like structure, placing the fluorophore and quencher in proximity. This hairpin opens in the presence of the target producing an increase in fluorescence (FIG. 1A). The proximity of the quencher to the fluorophore can result in reductions of fluorescent intensity of up to 98% (Marras et al. (2002) Nucleic Acids Res. 30(21):e122). The perceived efficiency can further be adjusted by altering the “stem strength” (which usually correlates with its % G&C content and length) which affects the number of beacons in the open state in the absence of the target. Accordingly, the tradeoff that a molecular beacon experiences is in regards to its stem strength, limiting either fluorescent increase upon hybridization or kinetics of hybridization (Yao and Tan 2004 Anal Biochem. 331(2):216-223). As shown in FIG. 1B, molecular beacons lose sensitivity by having low stem strength, which impacts both limits of detection and time to detection.

Molecular beacons have been used in many applications. Some in vitro applications include real-time monitoring of PCR products (Tyagi and Kramer 1996 Nat. Biotechnol. 14(3):303-308), sticky-end pairing (Li and Tan (2003) Anal. Biochem. 312(2):251-254), nuclease activity (Li et al. (2000) Nucleic Acids Res. 28(11):E52) and ligation rates (Tang et al. (2003) Nucleic Acids Res. 31(23):e148). One of the truly marvelous aspects of molecular beacons has been their ability to monitor real-time gene expression in vivo by targeting mRNA encoding sequences such as basic fibroblast growth factor (Matsuo (1988) Biochim. Biophys. Acta 1379(2):178-184), human c-fos (Tsuji et al. (2000) Biophys. J. 78(6):3260-3274), and β-actin (Perlette (2001) Anal. Chem. 73(22):5544-5550). Another pertinent application is biosensing. Biosensors have been developed for clinical diagnostics detecting pathogens such as HIV (Gonzalez et al. (1999) Proc. Natl. Acad. Sci. U.S.A. 96(21):12004-12009) and Francisella tularensis (Ramachandran et al. (2004) Biosens. Bioelectron. 19(7):727-736) with potential for developing bioterroism-sensing applications.

Molecular beacon aptamers are among the more recent adaptations of molecular beacons. Aptamers in general are structures that conform to a given shape, and typically refer to polynucleotide sequences used to target specific epitopes on polypeptides. They offer significant advantages in protein targeting over traditional peptide-antibody interactions, due to their lower state of free-energy in complex formation, the significantly smaller size of the aptamers, and the relative ease and low cost of replicating the polynucleotide sequences which compose most aptamers (Tombelli et al. (2005) Biosens. Bioelectron. 20(12):2424-2434).

Several versions of molecular beacon aptamers have been designed, the most straightforward of which was developed by Hamaguchi, and follows the conventional molecular beacon form of stem loop structure that melts in the presence of the target molecule (Hamaguchi et al. (2001) Anal. Biochem. 294(2):126-131). Others have targeted peptides such as thrombin and the TAT protein from HIV using quenching (e.g. the presence of the target causes the quencher and fluorophore to come together) or sandwiching (e.g. when a second nucleic acid sequence combines with the molecular beacon to sandwich the peptide) as means of detection (Yamamoto et al. (2000) Genes Cells 5(5):389-396; Li et al. (2002) Biochem. Biophys. Res. Commun. 292(1):31-40). Molecular beacon aptamers have the potential to be used in many similar applications to those currently using conventional molecular beacons. For example, they can be used for in vivo monitoring of protein expression and function, or for real-time monitoring of drug delivery, including cellular uptake and half-life. Despite their relative advantages, molecular beacons and molecular beacon aptamers are still not being used to their fullest extent. This is perhaps due to the relatively low cap on signal to background from the limitations on stem strength.

Regardless of the detection platform or strategy, the majority of biosensors incorporate molecular recognition through a biological affinity interaction. A biosensor cannot be more accurate than this interaction. This interaction is used for one or more functions that include identifying the presence of a given analyte, determining changes in expression level, and quantifying the agent (Call (2005) Crit. Rev. Microbiol. 31(2):91-99). Specificity and sensitivity in biosensor research often refer to the ability of the sensor to eliminate false positives and negatives, respectively, for one or more of the foregoing objectives. Unfortunately, there is usually a tradeoff between specificity and sensitivity, as shown in FIG. 2 (Bhanot et al. (2003) Biophys. J. 84(1):124-135).

Common methods of increasing sensitivity of an assay include reducing the noise in a system (Halperin et al. (2004) Biophys. J. 86(2):718-730; Nyholm (2005) Analyst 130(5):599-605), altering the geometry of the detection zone (Zarrin (1985) Analytical chemistry 57(13):2690; Chen and Dovichi (1994) J. Chromatogr. B Biomed. Appl. 657(2):265-269), increasing the signal either from amplified reporters like attaching more or stronger fluorophores or from amplified product as in PCR (Kuske et al. (2002) Appl. Environ. Microbiol. 64(7):2463-2472; Loge et al. (2002) Environ. Sci. Technol. 36(12):2754-2759), or increasing the affinity of the probe-target interaction (Reloglio et al. (2002) Nucleic Acids Res. 30(11):e51). Specificity is often achieved by lowering the probe affinity by altering the probe length or chemistry or by adding energy to the system such as increasing the reaction temperature (Lee et al. (2004) Nucleic Acids Res. 32(2):681-690; Letowski et al., (2004) J. Microbiol. Methods, 57(2):269-278). Adjusting sensitivity or specificity through these means leads to an endless cycle of tradeoffs that can never really improve test accuracy, which is a combined metric of specificity and sensitivity. While there are several methods that seem to offer gains without as much of a tradeoff (Liu et al. (2001) Environ. Microbiol. 3(10): 619-629; Dai et al. (2002) Nucleic Acids Res. 30(16):e86; Bhanot et al. (2003) Biophys J. 84(1):124-135; Tsourkas et al. (2003) Nucleic Acids Res. 31(4):1319-1330), these may have other potential difficulties with real-world matrices (Halperin et al. (2004) Biophys. J. 86(2):718-730). FIG. 2 illustrates tradeoffs seen in typical efforts to increase sensitivity.

In support of these limitations in improving biosensor accuracy, the numbers tell a compelling story. By way of identification of the presence of specific species, Peplies et al tested six strains of bacteria with a 1% rate of false positives and 41% rate of false negatives (Peplies et al. (2003) Appl. Environ. Microbiol. 69(3):1397-1407). Diagnostic polymerase chain reaction (PCR), although rarely having false negatives owing to its extreme sensitivity, is also able to detect virtually every trace contaminate and experiences a reported rate of false positives between 9 and 57% (Borst et al. (2004) Eur. J. Clin. Microbiol. Infect. Dis., 2004, 23(4):289-299). Detectors monitoring expression level are worse with identification of a change in expression from only 70-90% for samples above the sensitivity threshold and with a false positive rate of 10% (Draghici et al. (2004) Mil. Med. 169(8):654-659). While this rate of false positives and negatives may be damaging for phenotypic or other biological exploration, even one error can prove lethal in clinical diagnostics and could prove utterly devastating in homeland security applications.

Accordingly, there is a need to exploit the principles of cooperativity, as it is abundantly described in cell targeting applications (Mammen et al. (1998) Angew. Chem. Int. Ed. 37(20):2754-2794; Kiessling et al. (2000) Curr. Opin. Chem. Biol. 4(6):696-703; Fan and Merritt (2002) Curr. Drug Targets Infect. Disord. 2(2):161-167; Handl et al. (2004) Expert Opin. Ther. Targets 8(6):565-586), to combat the specificity-sensitivity tradeoff and to design more sensitive detection platforms. These principles have been mathematically described in cell targeting applications (Perelson, “Some mathematical models of receptor clustering by multivalent ligands,” in Cell Surface Dynamics: Concepts and Models, Perelson, A. S., et al. Ed., New York, Marcel Dekker, 223-276 (1984); Macken and Perelson, Branching Processes Applied to Cell Surface Aggregation Phenomena, Heidelberg, Springer-Verlag, (1985); Lauffenburger and Linderman, Receptors: Models for Binding, Trafficking, and Signaling, New York, Oxford University Press, (1993); Muller et al. (1998) Anal. Biochem. 261(2):149-158; Hubble (1999) Mol. Immunol. 36(1):13-18; Kitov and Bundle (2003) J. Am. Chem. Soc. 125(52):16271-16284; Huskens et al. (2004) J. Am. Chem. Soc. 126(21):6784-6797; Caplan and Rosca (2005) Ann. Biomed. Eng. 33(8):1113-1124). Cooperativity has also been shown to enhance single nucleotide polymorphism (SNP) detection and assay sensitivity (Gentalen and Chee (1999) Nucleic Acids Res. 27(6):1485-1491; Bates et al. (2005) Anal. Biochem. 342(1):59-68).

### SUMMARY

The present invention relates, in part, to uses of cooperativity to design biosensor detection strategies. By using rational design to predict enhanced kinetic performance and sensitivity, there is essentially no tradeoff between specificity and sensitivity in the design of cooperative assays. Increased resolving power is exhibited between detection limits for specific and nonspecific binding in such cooperative assays.

One aspect of the present invention provides for an algorithm for constructing cooperative interactions. Aspects of such cooperative interactions include, but are not limited to, increased specificity, sensitivity, accuracy, affinity and kinetics. Applications of the algorithm include, but are not limited to, design of tentacle probes, cooperative probe assays, drug constructs, cell targeting constructs, and synthetic antibodies.

Another aspect of the present invention pertains to cooperative probe assays (CPA). One aspect of CPA is the use of two or more probes to produce a cooperative interaction. One aspect of this cooperative interaction is the ability to produce enhanced sensitivity. Another aspect is the possibility to produce enhanced specificity. Other aspects includes, but are not limited by enhanced specificity, and sensitivity, without a tradeoff between the two. Yet another aspect is the ability to produce an increase in specificity and sensitivity simultaneously. Another aspect of CPA is the ability to apply CPA to a number of detection platforms, including, but not limited to, carbon nanotubes, surface plasmon resonance, laser-induced fluorescence, electrochemistry, mechanotransduction, and thermodetection. Applications of the CPA include, but are not limited to, diagnostics, biosensors, lab-on-a-chip, micro-total analysis systems, and other applications that are known by one skilled in the art.

In certain embodiments, the present invention provides a process of creating a cooperative probe assay for a target analyte. The process includes the steps of choosing an objective parameter in a cooperative binding assay system comprising one or more probes, modeling the thermodynamics and the dynamics of the cooperative binding assay to examine the effect of combining said probes on the objective parameter, and choosing a combination of probes to maximize the objective parameter. The objective parameter can be for example, kinetics, specificity, or sensitivity. In one aspect, the thermodynamics and the dynamics of the cooperative binding assay can be modeled by simultaneously solving a system of equations that describe the thermodynamic and dynamic state of the cooperative binding assay system, by applying an effective multivalent equilibrium constant arising from the equilibrium constants for one or more probes, targets, or complexes thereof.

In certain embodiments, the present invention provides a cooperative binding assay system for detecting an analyte comprising a cooperative probe. The cooperative probe comprises a probe set of two or more attached probes that are specific for separate regions of the target analyte. The probes can be directly or indirectly attached to each other.

The cooperative probe has one of or any combination (preferably 1 to 3) of the following characteristics: (i) an observed melting peak temperature that varies no more than about 10% with increasing concentration of the analyte when the concentration of analyte is greater than the concentration of the cooperative probe; (ii) a forward rate constant of a probe within the probe set that is greater than one and a half times its noncooperative forward rate constant value, (iii) an analyte binding affinity that is greater than one and a half times the sum of the noncooperative target analyte binding affinities of the individual probes for the target analyte, and (iv) at least one of the probes will not detectably bind to the analyte without the analyte binding to the other probes in the cooperative system. In certain aspects, the observed melting peak temperature of the cooperative probe varies no more than 8%, or no more than 5% with increasing concentration of the analyte when the concentration of analyte is greater than the concentration of the cooperative probe. In certain embodiments, the forward rate constant of at least one probe within the probe set is greater than 1.5, 2, 3, 4, 5, 8, 10, 15, 20, 50, 100, 300 or 1000 times its noncooperative forward rate constant value. In certain embodiments, the analyte binding affinity of the cooperative probe is greater than 1.5, 2, 3, 4, 5, 8, 10, 15, 20, 50, 100, 300 or 1000 times the sum of the noncooperative target analyte binding affinities of the individual probes for the target analyte.

In certain embodiments, the present invention provides a cooperative binding assay system for detecting an analyte while inhibiting non-specific detection of a variant of said analyte comprising an insertion sequence in the middle of said analyte, and where said cooperative binding assay comprises a probe set of two or more probes that are attached together wherein at least one of said probes is specific for said analyte and one of said probes is specific for said variant and having an observed melting peak temperature that varies no more than about 10% with increasing concentration of the variant analyte when the concentration of variant analyte is greater than the concentration of the cooperative probe. In certain aspects, the observed melting peak temperature of the cooperative probe varies no more than 8%, or no more than 5% with increasing concentration of the variant analyte when the concentration of variant analyte is greater than the concentration of the cooperative probe.

In certain embodiments of the present invention, a CPA method is provided for detecting an analyte in a biological or non-biological sample comprising the steps of: a. providing a first binding member and a second binding member, wherein the first binding member and the second binding member produce a signal in nonlinear proportion to the analyte, and wherein the first binding member and the second binding member are in proximity to each other for cooperative interactions with the target analyte; b. contacting the first binding member and the second binding member with the sample; c. providing an algorithm adapted for enhancing assay performance based on cooperativity between the two binding members to increase correlation between signal and analyte; and d. translating the signal into an analyte concentration or qualitative result. In this method, the cooperativity enhances an assay property selected from the group consisting of: faster kinetics, higher binding affinities, specificity, sensitivity, and a combination of specificity and sensitivity.

In another embodiment of the present invention, a cooperative probe assay system is providing for performing an assay to detect analyte in a biological or non-biological sample comprising: a. a capture probe; and b. a detection probe; wherein the capture probe and the detection probe each have a corresponding binding region for cooperative interactions with the target analyte to enhance assay performance.

In certain embodiments of the invention, tentacle probes are provided. When using any cooperative probe assay system, e.g., exemplary tentacle probes, of the present invention, the target analyte can be multiplied analyte or non-multiplied analyte. Non-multiplied analyte is analyte is not replicated by a primer-directed polymerase in the presence of the CPA system. However, the non-multiplied analyte can be amplified in the absence of the CPA system. After the completion of the amplification, the amplified analyte can then be analyzed using the CPA system. In certain embodiments of the CPA system, the cooperative probe will be a non-extendable probe. For example, in certain embodiments, the detection probe and/or capture probe of a tentacle probe will be non-extendable. In certain embodiments, the detection probe and/or capture probe will not be capable of initiating nucleic acid replication or amplification. In certain embodiments, the detection probe and/or capture probe will be blocked to prohibit polymerase catalyzed extension of the probe.

As such, the CPA system and tentacle probes, in particular, can be used in any amplification system, including real-time PCR. Preferably, the CPA system of the present invention provides enhanced kinetic performance, enhanced affinity, enhanced specificity and enhanced sensitivity over individual components of the cooperative probe. Exemplary tentacle probes of the present invention are one example of a CPA system and suitable to detect both a variety of target analytes which may or may not be multiplied or amplified in the presence of the probes. As used herein, the term “non-multiplied” refers to a target analyte that is not replicated by a primer-directed polymerase in the presence of either the detection probe or the capture probe. However, the non-multiplied analyte can be amplified in the absence of the CPA system prior to the analysis. The tentacle probes of the present invention, when used in combination with amplification, are not incorporated into either primer extension products or amplification products.

In preferred embodiments, one aspect of the tentacle probes of the present invention is their enhanced signal to background when compared to molecular beacons. Other aspects preferably include, for example, enhanced kinetic performance, enhanced affinity, enhanced specificity and enhanced sensitivity. The tentacle probes can possess one or all of these traits in addition to other enhancements over molecular beacons. Applications of the tentacle probes include, but are not limited to, diagnostics, amplification systems, including, for example, polymerase chain reaction (PCR), real-time PCR, strand displacement amplification (SDA), nucleic acid sequence based amplification (NASBA), transcription mediated amplification (TMA), the ligase chain reaction (LCR), rolling circle amplification, and RNA-directed RNA amplification catalyzed by an enzyme such as Q-beta replicase, biodetectors and sensors, and in vitro and in vivo monitoring of biological or chemical processes. Some such processes can include processes where two or more components interact or are desired to be observed. The tentacle probes can in such instances be used to observe the combination of components and their interaction in real time.

Tentacle probes of the present invention comprise a detection probe and a capture probe. The tentacle probes can comprise any combination of detection probes and capture probes. For example, a tentacle probe can have one detection probe and one capture probe, or one detection probe and two or more capture probes. In preferred embodiments, the detection probe is in an open conformation when bound to said target analyte and is in a closed conformation when not bound to said target analyte. The change in conformation generates a change in detectable signal. The detection probe comprises a first binding region and the capture probe comprises a second binding region that is different, i.e., distinct and separate, from the first binding region. In exemplary embodiments when the tentacle probes are used for detecting target analyte in a sample, the first and second binding region are specific for the target analyte.

The tentacle probes of the present invention can comprise a first arm region and a second arm region that form a stem duplex when the probe is in a closed conformation and are separated when the probe is in an open conformation. The arm regions can be attached to a signal altering moiety although it is not necessary for detection. In certain embodiments, one of the arm regions is attached to a signal altering moiety. In other embodiments, both of the arm regions are attached to signal altering moieties. The target binding region on the detection probe can be intermediate to said first and second arm region, although it need not be. In certain embodiments, the first or second arm will comprise all or part of the target binding region. In others, the first and second arm will comprise a part of the target binding region.

The capture probe can be attached to the detection probe directly or indirectly. The capture probe is attached to the detection probe in such way that the detection and capture probes can cooperatively interact with the target. In certain instances, the capture probe or the detection probe is independently non-extendable. In certain instances, the detection probe and/or capture probe will not be capable of initiating nucleic acid replication or amplification. In certain instances, the detection probe and/or capture probe will be blocked to prohibit polymerase catalyzed extension of the probe. In certain embodiments, the target analyte is single stranded or double stranded nucleic acid and the capture probe and the detection probe comprise a sequence that is complementary to the same stand of nucleic acid and that is present on non-multiplied nucleic acid.

The first and second signal altering moieties can be members of a energy transfer pair although they need not be. The tentacle probe can use other mechanisms besides energy transfer for signaling the presence of analyte, including, for example an enzyme and an enzyme inhibitor pair. The tentacle probe can also use fluorescent polarization as a signaling mechanism. The signal altering moieties can be attached directly or, indirectly, e.g., via linkers, to the correspondent arms.

The first and second signal altering moieties can be members of a fluorescence energy transfer pair. In certain embodiments, the first signal altering moiety will be a fluorophore and the second altering moiety will be a fluorescence quencher. In the absence of the target analyte, the detection probe is predominantly in the closed conformation, in which the fluorophore and the quencher are in close proximity for effective energy transfer, thus fluorescent signal is effectively quenched. In the presence of the target analyte, the detection probe binds to the target analyte, resulting in the open conformation. In this conformation, the fluorophore and the quencher are separated and a fluorescent signal is emitted for detection.

In still another embodiment, the tentacle probe further comprises a linking moiety to assist the attachment of the tentacle probe to a solid phase for a solid phase assay. The linking moiety can be connected to the capture probe or the detection probe or both.

In certain embodiments, the tentacle probe is adapted to perform a particular purpose. For example, in certain embodiments, the tentacle probe will be for analyzing and/or identifying a target nucleic acid in a sample or for analyzing and/or identifying a target nucleic acid in a sample while inhibiting detection of a variant of said nucleic acid comprising an insertion sequence. For example, in certain embodiments, instead of the detection probe and capture probe having a binding sequence specific for the same target analyte, the detection probe will comprise a binding sequence that is complementary to a sequence present on the target analyte but not on the variant (i.e., it will be complementary to a sequence that is disrupted by the insertion) and the second binding region will comprise a sequence that is complementary to the insertion sequence on the variant. In other embodiments, the detection probe and capture probe will have a binding sequence specific that is complementary to a sequence present on the target analyte and a linker linking the detection and capture probe will comprise a sequence that is complementary to the insertion sequence on the variant.

In some embodiments, cooperativity of the assay can be used to overcome certain secondary structure present in the analyte. One probe within the cooperative pair binds with a region with low secondary structure within the vicinity of the region containing large secondary structure and enhances the kinetics of binding to opening the secondary structure and resulting in a detection. In other embodiments, one of the probes in the cooperative set binds to a region adjacent to and including the secondary structure, causing it to open up. In other embodiments these methods can be applied to overcoming tertiary or quaternary structure binding limitations.

Although the capture probe can be, for example, a linear probe that always remains in the same conformation whether bound or not bound to a target analyte, it can also, in certain embodiments, look very much like a detection probe. It can be in an open conformation when bound to a target analyte and in a closed conformation when not bound to a target analyte. The change in conformation can also, if desired, generate a change in detectable signal. The capture probe can, if desired, comprise a first arm region and a second arm region that form a stem duplex when the probe is in a closed conformation and are separated when the probe is in an open conformation. If desired, the arm regions can be attached to a signal altering moiety. One or both of the arm regions can be attached to a signal altering moiety. The target binding region on the detection probe can be intermediate to said first and second arm region. Alternatively, the first or second arm can comprise all or part of the target binding region or the first and second arm can comprise a part of the target binding region

The present invention provides methods for using a cooperative probe of the present invention, including a tentacle probe, for analyzing and/or identifying a target analyte in a sample suspected of containing the analyte, including detecting the absence or presence of the target analyte in the sample. The method comprises the steps of contacting the cooperative probe with the sample; and measuring the signal. The methods of the present invention can be used for analyzing and identifying a single target analyte in a sample or multiple target analytes in a sample simultaneously.

When the methods of the present invention are performed in a multiplexing format, two or more cooperative probes, each with a distinguishable detectable signal and/or specific for different analytes, can be employed. For example, the present methods can be used to detect two or more different analytes in a sample. In certain exemplary methods, two or more tentacle probes comprising binding sequences that bind to different analytes, and comprising distinguishable detectable signals can be employed. Accordingly, it will be understood that a first binding region on one tentacle probe can be different than a first binding region on a second tentacle probe. A second binding region on one tentacle probe can be different than a second binding region on a second tentacle probe. Similarly, a first and second signal altering moiety on one tentacle probe can be different from a first and second signal altering moiety on a second tentacle probe. Another example is where tentacle probes specific to one target can be localized in different locations than tentacle probes specific to another target. The methods of the present invention can also be used in combination with amplification systems. For example, the tentacle probe can be contacted with the test sample during an amplification reaction or after an amplification reaction.

The capture probes and detection probes of the present invention are not meant to function as primers. In certain embodiments, the detection and/or capture probe not only will not function as a primer but will be incapable of initiating nucleic acid replication or amplification. In certain embodiments, this will be because the detection probe and/or capture probe of a tentacle probe will be non-extendable. In certain embodiments, the detection probe and/or capture probe will be blocked to prohibit polymerase catalyzed extension of the probe. In certain embodiments, the capture and detection probe will bind to a region of the target nucleic acid that is outside of the primer binding sites of the target nucleic acid.

In certain embodiments, the target analyte is single stranded or double stranded nucleic acid and the capture probe and the detection probe comprise a sequence that is complementary to the same stand of nucleic acid and that is present on non-multiplied nucleic acid.

The present invention further relates to a kit for analyzing and identifying a target analyte in a sample. The kit comprises one or more cooperative probes of the present invention. The kit can also comprise instructions on their use. When used in a amplification reaction, the kit can further comprise amplification reagents.

In certain embodiments, a tentacle probe of the present invention will comprise a detection probe comprising a first target binding region comprising the sequence TGG CGG AAA AGC TAA TAT AGT AA (SEQ ID NO:2), a first arm region attached to a first signal altering moiety and a second arm region attached to a second signal altering moiety wherein said first arm region and second arm region are complementary to each other; and at least one capture probe comprising a second target binding region comprising the sequence GAT TAA AAT GTC CAG TGT ACC AG (SEQ ID NO:3); wherein the capture probe is attached to the detection probe directly or indirectly. In certain aspects, the first arm comprises the sequence gccac (SEQ ID NO:6) and the second arm region comprises the sequence gtggc (SEQ ID NO:5). In certain aspects, the first arm comprises the sequence cgccac (SEQ ID NO:9) and the second arm region comprises the sequence gtggcg (SEQ ID NO:8). In certain aspects, the first arm comprises the sequence ccgccac (SEQ ID NO:12) and the second arm region comprises the sequence gtggcgg (SEQ ID NO:11). In certain aspects, the first arm comprises the sequence ccgccacc (SEQ ID NO:15) and the second arm region comprises the sequence ggtggcgg (SEQ ID NO:14). In certain aspects, the first arm comprises the sequence ccgccaccc (SEQ ID NO:18) and the second arm region comprises the sequence gggtggcgg (SEQ ID NO:17).

In certain embodiments, a tentacle probe of the present invention will comprise a detection probe comprising a first target binding region comprising the sequence CTTCTACGCATGACCATATTC (SEQ ID NO:37), and at least one capture probe comprising a second target binding region comprising the sequence ATAAAGGGAAAGTATACCG (SEQ ID NO:25), wherein the capture probe is attached to the detection probe directly or indirectly. In certain aspects, the tentacle probe will have a first arm region attached to a first signal altering moiety and a second arm region attached to a second signal altering moiety wherein said first arm region and second arm region are complementary to each other. In certain aspects, the first arm comprises the sequence CTTCTACGC (SEQ ID NO:27) which is also part of the detection sequence and the second arm comprises the sequence GCGTAGAAG (SEQ ID NO:28).

In certain embodiment, the present invention will provide a kit comprising a detection probe comprising a first target binding region comprising the sequence CTTCTACGCATGACCATATTC (SEQ ID NO:37), and at least one capture probe comprising a second target binding region comprising the sequence ATAAAGGGAAAGTATACCG (SEQ ID NO:25), wherein the capture probe is attached to the detection probe directly or indirectly. In certain aspects, the tentacle probe will have a first arm region attached to a first signal altering moiety and a second arm region attached to a second signal altering moiety wherein said first arm region and second arm region are complementary to each other. In certain aspects, the first arm comprises the sequence CTTCTACGC (SEQ ID NO:27) which is also part of the detection sequence and the second arm comprises the sequence GCGTAGAAG (SEQ ID NO:28). In certain embodiments, the kit will further comprise the forward primer BAGYRA1614F [5′-GGG AAC AAA TGA TGA TGA TTT CGT-3′] (SEQ ID NO:29) and the reverse primer BAGYRA1732R [5′-ACT CTG GGA TTT CAT ATC CTT TCG T-3′] (SEQ ID NO:30). In certain embodiments, a tentacle probe of the present invention will comprise a detection probe comprising a first target binding region comprising CGA GGT TCA GGT GAG CAC G (SEQ ID NO:38), and at least one capture probe comprising a second target binding region comprising GAG TAT TCG TCT GGG GG (SEQ ID NO:31); wherein the capture probe is attached to the detection probe directly or indirectly. In certain aspects, the tentacle probe will have a first arm region attached to a first signal altering moiety and a second arm region attached to a second signal altering moiety wherein said first arm region and second arm region are complementary to each other. In certain aspects, the first arm comprises the sequence CCC CGA G (SEQ ID NO:33) which is also part of the detection sequence and the second arm comprises the sequence CT CGGGG (SEQ ID NO:34).

In certain embodiment, the present invention will provide a kit comprising a tentacle probe of the present invention will comprise a detection probe comprising a first target binding region comprising CGA GGT TCA GGT GAG CAC G (SEQ ID NO:38), and at least one capture probe comprising a second target binding region comprising GAG TAT TCG TCT GGG GG (SEQ ID NO:31); wherein the capture probe is attached to the detection probe directly or indirectly. In certain aspects, the tentacle probe will have a first arm region attached to a first signal altering moiety and a second arm region attached to a second signal altering moiety wherein said first arm region and second arm region are complementary to each other. In certain aspects, the first arm comprises the sequence CCC CGA G (SEQ ID NO:33) which is also part of the detection sequence and the second arm comprises the sequence CT CGGGG (SEQ ID NO:34). In certain embodiments, the kit will further comprise the forward primer [5′-gcaggaaatgcgcaatgc-3′] (SEQ ID NO:35) and the reverse primer [5′-gggcggatccccacttta-3′] (SEQ ID NO:36).

Other aspects of the present invention are described throughout the specification.

### BRIEF DESCRIPTION OF THE DRAWINGS

FIG. 1A-B: Molecular beacon hybridization and kinetic-sensitivity tradeoff. (A) In the presence of the target polynucleotide, the hairpin structure opens, causing a detectable increase in fluorescence. (B) The sensitivity affects the percent equilibrium necessary for detection, which affects the time to detection by orders of magnitude. Molecular beacons lose sensitivity by having low stem strengths, impacting both limits of detection and time to detection. In FIG. 1B, the percent (%) of equilibrium that is depicted, from upper right to lower left, is 98%, 1% and 0.01%, respectively.

FIG. 2A-B: Sensitivity versus Specificity: (A) shows the effect of increasing sensitivity by increasing the signal to noise ratio on a Langmuir isotherm for specific and nonspecific binding. While improving the signal to noise ratio increases the sensitivity of the system, specificity is sacrificed; (B) shows the effect of increasing sensitivity by increasing probe affinity. Sensitivity still occurs at the sacrifice of specificity. An increase in specificity causes the opposite effect with a loss of sensitivity. (In FIG. 2A, the “Perfect match” is depicted as the upper left curve, and the “SNP” is depicted as the lower right curve. In FIG. 2B, the curves from upper left to lower right depict: “High Affinity Perfect match”, “High Affinity SNP”, “Low Affinity Perfect match” and “Low Affinity SNP”, respectively. The y-axis shows the fractions of probes bound and the x-axis shows the analyte concentration.)

FIG. 3: Tentacle Probes function similarly to molecular beacons except the presence of a capture region allows additional pathways. In the lower left, the probe (P) and target (T) can interact forming a hybrid with either the detection probe (C_{det}) or the capture probe (C_{cap}). Once the first binding event occurs, a second binding event can occur at a much accelerated rate over the free solution rate due to the enhanced local concentration, forming a hybrid with both detection probes (C_{both}). The equilibrium constants together with effective equilibrium constants for each state are shown between the states.

FIG. 4A-B. Two demonstrations of increase in local probe concentration for 2^{nd} binding event. (A) shows a polynucleotide target that could conceivably bind with many different probes in a large area, creating a variable local probe concentration. (B) shows a polypeptide which is rather large compared to the binding aptamers, creating a local probe concentration which is constant (e.g. always one free aptamer, no more and no less, available for the 2^{nd} binding event in a small, constant volume).

FIG. 5: Diagram of variables in equations. This Figure shows possible interactions between reagents (target and probes A and B) and products (complex of target and probes A, B or both A and B). These definitions for variables also work for nucleic acid hybridizations.

FIG. 6A-D: Increases in specificity for cooperative probe assays over standard linear probes. These images demonstrate predicted improvements in single nucleotide polymorphism detection as a function of affinity and target concentration. FIGS. 6A and 6C are continuous flow sensors and show equal improvement at different probe concentrations (top curve with a probe concentration of 1 μM, bottom curve with a probe concentration of 1 nM), in contrast to batch sensors (FIGS. 6B and 6D) where improvement changes with both target and probe concentration. (In FIGS. 6A and 6C, T=1e-12, 1e-10, 1e-8 and 1e-6 from top to bottom. The same order is depicted in FIGS. 6B and 6D, except that in FIG. 6B, the lowest concentrations and the highest concentrations overlap, and in FIG. 6D, 1e-10 and 1e-12 overlap in the top curve.)

FIG. 7A-C: Tentacle probe embodiments. (A) shows one embodiment of the tentacle probe achieved by mixing 1:1 ratio of the detection probe and the capture probe (dotted line) prior to spotting. Its disadvantage is a minimum distance between probes of around 7-9 nm (10¹² probes cm⁻²). (7B) shows an exemplary detection probe covalently attached to the capture probe (dotted line), placing the two probes in close proximity. (7C) shows an exemplary detection probe/capture probe complex in a branched configuration. This chemistry is ideal due to the close proximity of the capture probe to the beacon and its distance from the biosensor surface.

FIG. 8A-C: Cooperative probe assay embodiments. (8A) shows a simple embodiment of the CPA achieved by mixing 1:1 ratio of the two different probes (dashed and dotted lines.) Its disadvantage is a minimum distance between probes of around 7-9 nm (10¹² probes cm⁻²). (8B) shows two probes attached in a linear fashion to the same molecule. (8C) shows a two probe complex in a branched configuration. This chemistry is ideal due to the close proximity of the probes to each other and their distance from the biosensor surface.

FIG. 9. Characterization of tentacle probes. This figure shows thermal denaturation profiles of an exemplary tentacle probe (dotted line) and the hybrid formed between the tentacle probe and its oligonucleotide target (dashed line). The profiles indicate that this tentacle probe can be used below 55° C.

FIG. 10. Tentacle probe design and function. This figure shows one example of an interaction of an exemplary tentacle probe with a target analyte, resulting in a conformational change in the tentacle probe from a closed state to an open state and thus yielding a fluorescence signal.

FIG. 11. Differentiation between wild-type and single nucleotide polymorphism. This figure shows detection of wild-type analyte on the left and non-detection of the wild-type having a single nucleotide polymorphism.

FIG. 12A-B. Mechanism of tentacle probes in qPCR. (12A) shows mechanism of tentacle probes in qPCR with exonuclease active polymerase. The upper diagram shows detection of wild-type analyte the lower diagram shows non-detection of the wild-type having a polymorphism. (12B) shows mechanism of exonuclease deficient polymerase chain reaction with tentacle probes. The upper diagram shows detection of wild-type analyte the lower diagram shows non-detection of the wild-type having a polymorphism.

FIG. 13. Binding Rate This figure shows that tentacle probes (TP) are 100 to 200 times faster in hybridization reactions for stem lengths from 5 to 9 than Molecular Beacons (MB) with the same stem strengths.

FIG. 14A-B. Specificity This figure shows that tentacle probes require the presence of a match to both the capture and detection probes in order to produce a strongly detectable signal. Whereas molecular beacons do not have the same level of specificity, reporting false positives to analyte that only matches the detection region. 14A shows specificity for an exemplary tentacly probe. 14B show specificity for a molecular beacon.

FIG. 15A-D. Effect of the linker on the melting temperature of capture probes. (15A) Exemplary tentacle probes with high capture probe affinity exhibit melting curves that do not shift with concentration (upper left) and lead to high specificities. This also leads to what appears to be binding penalties in gradual slope. 15B, 15C and 15D all used probes with relatively weak capture probes. By using weak capture probes, melting curves begin to shift and appear to lose the binding penalties. Probes in the 15B used no linker. 15C and 15D had Tentacle Probes with the same length linker (about 3.06 nm), one carbon and one PEG. Although visually it is hard to distinguish these three graphs, overlaying them (not shown) reveals the PEG and carbon linker are virtually identical. They differ from the no linker example by about a 1 deg C. shift in the melting temperatures. It appears, therefore, that linker composition does not dramatically affect binding properties of Tentacle Probes.

FIG. 16. PCR Applications of tentacle probes. This figure shows an exemplary method using PCR with an exemplary tentacle probe.

FIG. 17. PCR Applications of tentacle probes. This figures shows discrimination of bacillus anthracis from bacillus cereus in gyrA gene, which differs by a SNP in region of detection. Discrimination is performed by presence or absence of a signal only in contrast with normal methods comparing ratio of signals. Concentrations from 20 copies to 20,000 copies of b. anthracis were detected. Concentrations tested up to 20,000 copies of b. cereus were not distinguishable from the background even after 95 cycles of amplification. This experiment was carried out with exonuclease active Taq polymerase in a manner similar to Taqman probes. Cycle thresholds were within 1 to 2 cycles of Taqman probes (not shown). Bacillus Cereus amplification was verified by gel electrophoresis.

FIG. 18. Application of tentacle probes. This figure shows melting peaks and curves between tentacle probe and y. pestis (Solid line) and y. pseudotuberculosis (Dot line). Using an exemplary tentacle probe, there is a definite window between about 68 and 70° C. where specific binding is detectable, but nonspecific binding is not. After determining the proper temperature for monitoring fluorescence from these melting curves, qPCR was performed for specific identification of y. pestis (FIG. 19). The same advantage of fluorescence monitoring at higher temperatures is not available for MGB Taqman probes because they are digested at the primer annealing temperature.

FIG. 19A-B. Comparison of MGB Tagman vs. tentacle probes. 19A This figure shows that MGB Taqman has been unable to distinguish y. pestis from y. pseudotuberculosis at the insertion in the yp48 gene. False positives (open squares) on LC 4.0 occur around 3 cycles after detection of y. pestis (diamonds) as seen in figure on left. 19B In contrast, Tentacle Probes (right) experience 0% false positives at concentrations tested up to 20,000 copies of y. pseudotuberculosis even after 95 cycles of amplification. 95% confidence intervals are included. Tentacle Probes required approximately 4 to 5 extra cycles for detection. This experiment was performed with exonuclease deficient polymerase. It is believed that the longer cycle detection times are due to high probe melting temperatures reducing the efficiency of amplification. Repeats of the experiment with exonuclease active polymerase resulted in similar cycle threshold for both TP and MGB Taqman. Alternatively, probes can be designed with lower melting temperatures to reduce the cycle threshold.

FIG. 20. Rate Constants. This figure shows rate constants for the different stem lengths for exemplary tentacle probes (dark bars) and molecular beacons (light bars).

FIG. 21A-B. Fitted Melting Curves. 21A shows molecular beacon melting curves with data (7 base stem in 500 nM SNP target). 21B shows exemplary tentacle probe(8 base stem in 5 μM wild type target) fitted melting curves with data (8 base stem in 5 μM wild type target). Squares are with target, triangles are probes only.

FIG. 22A-B. Bound Probes. These graphs show the log plot of the fraction of probes bound by wild type (filled square) and SNP targets (open triangles) in 1 μM concentrations as a function of temperature. Fitted curves are also displayed for wild type (solid line) and SNP containing analyte (dashed line). 22A shows molecular beacon binding and 22B shows tentacle probe binding.

FIG. 23. Melting Curves. This figure shows melting curves for an exemplary tentacle probe for discrimination and localization of SNP's within the detection probe with 500 nM of each target type.

FIG. 24A-B. Melting Curves. These figures shows melting curves for an exemplary tentacle probe (24A) and molecular beacon (24B) for wildtype and SNP_{del} analyte concentrations from 50 nM to 50 μM.

FIG. 25A-B.Isotherms. These figures show isotherms of wild type binding (solid diamonds) and SNP binding (open triangles) as a function of target concentration performed at 60° C. (TP, FIG. 25A) and 55° C. (MB, FIG. 25B). Theoretical predictions (solid line—WT, dashed line—SNP) are produced from thermodynamic constants extracted from melting curves and are plotted against experimental data. 95% confidence intervals are shown for each data point but are not visible on higher binding values because of the log axis. Lower confidence intervals do not appear on some data points because they include zero. The horizontal line is the detection threshold set at one standard deviation over background and shows that even with this sensitive threshold, SNP's do not cause false positives for Tentacle Probes even at millimolar concentrations.

FIG. 26. Detection Strategies. This figures shows detection of wild-type analyte by molecular beacon (left), exemplary tentacle probe TP1 (middle), exemplary tentacle probe TP2 (right). Tentacle probe 1 has a capture probe that comprises a sequence complementary to a nonspecific insertion. Tentacle probe 2 has a linker that comprises a sequence complementary to a nonspecific insertion.

FIG. 27. Detection Strategies. This figure shows that the insertion can form a hairpin-like structure forming an exact match to the molecular beacon and contributing to false positives (left). Exemplary tentacle probe TP1 has a capture probe that comprises a sequence complementary to a nonspecific insertion and forms a double helix with the insertion region preventing nonspecific analyte from forming a hairpin and matching detection probe (middle.) Exemplary tentacle probe 2 has a linker that comprises a sequence complementary to a nonspecific insertion and forms a double helix with the insertion, preventing the detection probe from doubling back and hybridizing with non-specific analyte.

FIG. 28. Alternative tentacle probe designs. This figures shows some alternative tentacle probe designs wherein the tentacle probes have multiple detection probes for cooperative interactions.

FIG. 29. Alternative tentacle probe designs. This figures shows some alternative tentacle probe designs. The tentacle probe on the left has a capture and detection probe, but the detection probe is not contiguous, possessing a target binding region attached to a stem with a signal altering moiety via a linker. The tentacle probe on the left has a stem that is not attached to the detection probe through any means beside an affinity interaction. In the presence of analyte, the stem is released into free solution.

FIGS. 30A-B. Boil preps of 20 environmental samples of various strains of b. cereus and b. thuringensis were run for TaqMan-MGB (30A) and tentacle probes (30B). TaqMan-MGB experienced 21 false positives out of 29 samples. Tentacle probes had no false positives.

### DETAILED DESCRIPTION OF THE INVENTION

As used in this disclosure, the singular forms “a”, “an”, and “the” may refer to plural articles unless specifically stated otherwise. Thus, for example, references to a method of manufacturing, derivatizing, or treating “an analyte” may include a mixture of one or more analytes. Furthermore, the use of grammatical equivalents such as “nucleic acids”, “polynucleotides”, or “oligonucleotides” are not meant to imply differences among these terms unless specifically indicated.

To facilitate understanding of the invention set forth in the disclosure that follows, a number of terms are defined below.

### DEFINITIONS

The term “amplicon” refers to a nucleic acid product generated in an amplification reaction.

The term “amplification” refers to the process in which “replication” is repeated at least once, and preferably more than once in a cyclic process such that the number of copies of the nucleic acid sequence is increased in either a linear or logarithmic fashion.

The term “complementary strand” refers to a nucleic acid sequence strand which, when aligned with the nucleic acid sequence of one strand of the target nucleic acid, such that the 5′ end of the sequence is paired with the 3′ end of the other sequence in antiparallel association, forms a stable duplex. Complementarity need not be perfect. Stable duplexes can be formed with mismatched nucleotides.

The terms “detect” or “detection” or “detecting the presence or absence of an analyte” or “measuring the signal” refers to a process to provide qualitative or quantitative information about an analyte. The phrase “measuring the signal” is meant to include any method of measuring signal including a simple observation of a change in signal.

The term “label” refers to any atom or molecule that can be attached to a molecule for detection.

The terms “peptide”, “polypeptide”, “oligopeptide”, or “protein” refers to two or more covalently linked, naturally occurring or synthetically manufactured amino acids. There is no intended distinction between the length of a “peptide”, “polypeptide”, “oligopeptide”, or “protein”.

The term “peptide nucleic acid” or “PNA” refers to an analogue of DNA that has a backbone that comprises amino acids or derivatives or analogues thereof, rather than the sugar-phosphate backbone of nucleic acids (DNA and RNA). PNA mimics the behavior of a natural nucleic acid and binds complementary nucleic acid strands.

The terms “polynucleotide”, “oligonucleotide” or “nucleic acid” refer to polydeoxyribonucleotides (DNA), polyribonucleotides (RNA), analogs and derivatives thereof. There is no intended distinction between the length of a “polynucleotide”, “oligonucleotide” or “nucleic acid”.

The term “primer” refers to an oligonucleotide that functions to initiate the nucleic acid replication or amplification process.

The term “probe” generally refers to a molecule having a desired affinity towards a target analyte. It can be an oligonucleotide in the broad sense, by which is meant that it can be DNA, RNA, a mixture of DNA and RNA, and it can include non-natural nucleotides and non-natural nucleotide linkages. It can also be a molecule other than oligonucleotide, such as, for example, an amino acid, sugar, lectin, peptide, and the like. A probe functions in part by bonding to a target analyte in a reaction mixture. Generally, a probe comprises a binding region that is capable of binding to an intended target region.

The term “target” refers to the analyte which a probe is designed to bind. In some embodiments, the target is the analyte which is being detected. In other embodiments, the target is a variant of the analyte which is being detected and is bound to inhibit its detection and/or amplification.

The term “target binding region” refers to the region on the detection probe or capture probe that is capable of binding to the target of interest. In certain embodiments, a probe will comprise a binding region that is single-stranded oligonucleotide that can hybridize to its intended target sequence (or sequences) at the detection temperature (or temperatures) to generate detectable signals, such as fluorescence. Probes that are very specific for a perfectly complementary target sequence and strongly reject closely related sequences having one or a few mismatched bases are “allele discriminating.” Probes that hybridize under at least one applicable detection condition not only to perfectly complementary sequences but also to partially complementary sequences having one or more mismatched bases are “mismatch tolerant” probes. The detection probe and/or capture probes of the present invention can be designed to be mismatch tolerant or allele discriminating.

Hybridization can occur under conditions of high stringency (also called “stringent hybridization conditions”), moderate stringency, or low stringency. “Stringent hybridization conditions” are conditions under which a probe will hybridize to its target subsequence, typically in a complex mixture of nucleic acid, but not to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions can be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide. For high stringency hybridization, a positive signal is at least two times background, preferably 10 times background hybridization.

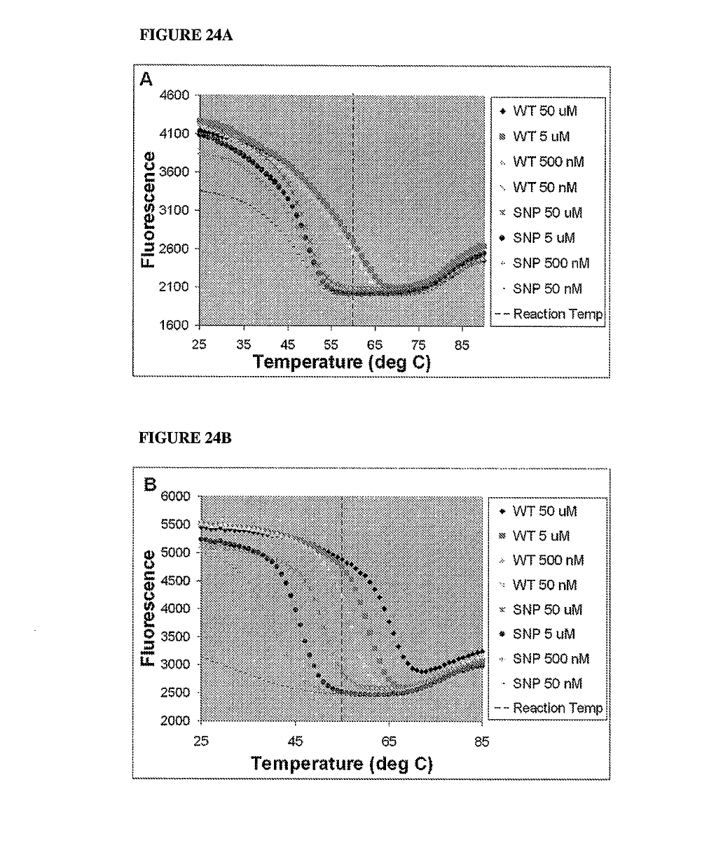

Another method to create highly stringent conditions is to generate a melting curve of the probe with target analyte and with near neighbors. The temperature at which target analyte still remains bound to the probe, but near neighbors are melted off is the desired temperature for high stringency reaction conditions. For example, looking at the melting curves for a tentacle Probe in FIG. 24a, an exemplary high stringency reaction condition would be 60° C., where SNP possessing analyte at multiple concentrations have melted, but wild type target at multiple concentrations is still bound.

Examples of moderate stringency are as follows: Melting curves are generated as described with high stringency conditions. However, the reaction temperature used can be slightly lower to accommodate single base mutations, insertions or deletions. Salt conditions and organic solvents can be added or changed in order to shift the melting curves. For example, looking at the melting curves for a Tentacle Probe in FIG. 24a, an exemplary moderate stringency reaction condition would be 45° C., where SNP possessing analyte and wild type analyte at multiple concentrations are bound, but greater numbers of mutations would be expected to melt.

Examples of low stringency are as follows: Melting curves are generated as described with moderate stringency conditions. However, the reaction temperature used can be slightly lower to accommodate multiple base mutations, insertions or deletions. Salt conditions and organic solvents can be added or changed in order to shift the melting curves. The length or affinity of the probes for the target analyte can also be increased in order to shift melting curves. For example, looking at the melting curves for a Tentacle Probe in FIG. 24a, an exemplary low stringency reaction condition would be room temperature, where SNP possessing analyte and wild type analyte at multiple concentrations are bound tightly, and where greater numbers of mutations would be expected to bind as well. This type of low stringency could be useful for identifying highly polymorphic targets such as HIV or for identifying classes of targets such as all bacteria in the bacillus cereus group.

A detection and/or capture probe can, comprise an aptamer that can bind to its intended target. The term “aptamer” refers to a nucleic acid molecule that is capable of binding to a particular molecule of interest with high affinity and specificity (Tuerk and Gold, Science 249:505 (1990); Ellington and Szostak, Nature 346:818 (1990)). The binding of a ligand to an aptamer, which is typically RNA, changes the conformation of the aptamer and the nucleic acid within which the aptamer is located. The conformation change inhibits translation of an mRNA in which the aptamer is located, for example, or otherwise interferes with the normal activity of the nucleic acid. Aptamers may also be composed of DNA or may comprise non-natural nucleotides and nucleotide analogs. An aptamer will most typically have been obtained by in vitro selection for binding of a target molecule. However, in vivo selection of an aptamer is also possible.

The term “replication” refers to the process in which a complementary strand of a nucleic acid strand is synthesized by a polymerase enzyme. In a “primer directed” replication, this process generally requires a hydroxyl group (OH) at the 3′ end of (deoxy)ribose moiety of the terminal nucleotide of a duplexed “primer” to initiate replication.

The term “single nucleotide polymorphism” (SNP) refers to a single-bases variation in the genetic code.

The term “variant” or “mutant” analyte refers to an analyte that is different than its wildtype counterpart.

The term wildtype as used herein refers to the typical form of an organism, strain, gene, or characteristic as it occurs in nature, as distinguished from mutant or variant forms that can result from selective breeding.

The term “cooperativity” refers to the use of two or more probes in a set, where a binding event to one probe results in the presentation of bound analyte at an enhanced local concentration to a second probe, resulting in increases in kinetics, affinity, sensitivity and/or specificity of the reaction over what the second probe or set of probes would experience in a noncooperative setting such as in free solution. Cooperativity can refer to enhanced characteristics contributing to the identification of an analyte or the inhibition of identification of an analyte. A cooperative probe is one that has two or more probes in close proximity that act cooperatively.

The term “tentacle probe” refers to a type of cooperative probe having a detection probe and a capture probe wherein the detection probe can change conformation and the change in conformation generates a change in detectable signal. In general, upon binding to a target analyte, the interactions between the detection probe and the target analyte shifts the equilibrium predominantly towards to an open conformation.

A “small organic molecule” is a carbon-containing molecule which is typically less than about 2000 daltons. More typically, the small organic molecule is a carbon-containing molecule of less than about 1000 daltons. The small organic molecule may or may not be a biomolecule with known biological activity.

### Cooperativity

Several models have been designed to exemplify the present inventions. Although the model used throughout the discussion is generally based on the interaction of nucleotides as binding members, it should be understood that this model is easily adapted for any type of binding reaction, such as that between an aptamer and a polypeptide epitope, a ligand and a receptor, and the like.

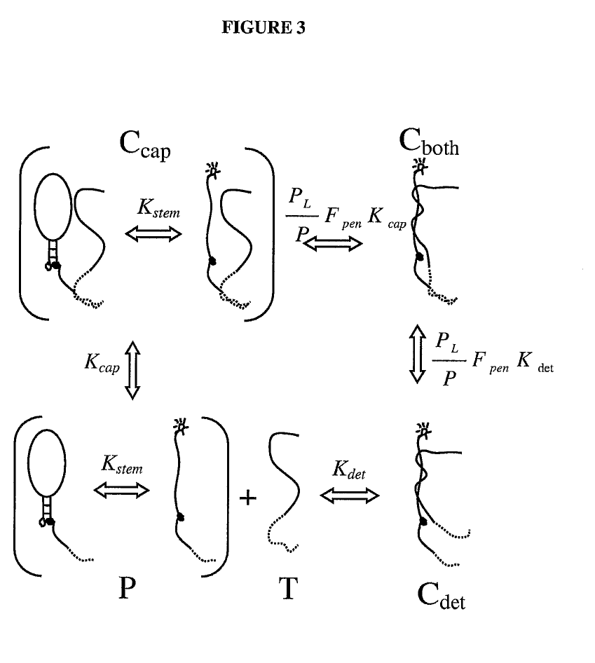

The first model is a mechanistic model of a tentacle probe having a single capture probe (FIG. 3). While FIG. 3 depicts the general form of cooperative interaction, it must be understood that there are a number of embodiments for attaching a detection probe and a capture probe. FIG. 3 demonstrates the possible states of cooperative binding, neglecting aggregation through crosslinking.

Tentacle probe (TP) technologies are optimized for sensitivity for both polynucleotide and polypeptide detection by exploiting cooperativity. Similar principles of cooperative binding are applied for enhanced specificity without a tradeoff in cooperative probe assays (CPA) for use with many detection platforms. CPA typically relies on two or more probe binding events to increase specificity and sensitivity. It achieves its enhanced avidity (effective affinity), which is the cause of increased assay accuracy, from the kinetics of the second binding event.

The physics of binding can be expressed by the following equations and then used to optimize the increase in specificity and sensitivity of CPA over standard linear probe assays.

$$\begin{matrix}\frac{\partial {C}_{A}}{\partial t} = {k}_{f , A} \cdot {P}_{A} \cdot T - {k}_{r , A} \cdot {C}_{A} - {k}_{f , B} \cdot {P}_{s , B} \cdot {C}_{A} + {k}_{r , B} \cdot {C}_{AB} & ( 1 ) \\ \frac{\partial {C}_{B}}{\partial t} = {k}_{f , B} \cdot {P}_{B} \cdot T - {k}_{r , B} \cdot {C}_{B} - {k}_{f , A} \cdot {P}_{s , A} \cdot {C}_{B} + {k}_{r , A} \cdot {C}_{AB} & ( 2 ) \\ \frac{\partial {C}_{AB}}{\partial t} = {k}_{f , A} \cdot {P}_{s , A} \cdot {C}_{B} + {k}_{f , B} \cdot {P}_{s , B} \cdot {C}_{A} - {k}_{r , A} \cdot {C}_{AB} - {k}_{r , B} \cdot {C}_{AB} & ( 3 ) \\ \frac{\partial T}{\partial t} = - {k}_{f , A} \cdot {P}_{A} \cdot T + {k}_{r , A} \cdot {C}_{A} - {k}_{f , B} \cdot {P}_{B} \cdot T + {k}_{r , B} \cdot {C}_{B} & ( 4 )\end{matrix}$$

The Equations that follow demonstrate how the differential equations above were used to create Tentacle Probes and can be used to create other exemplary cooperative probe assays.

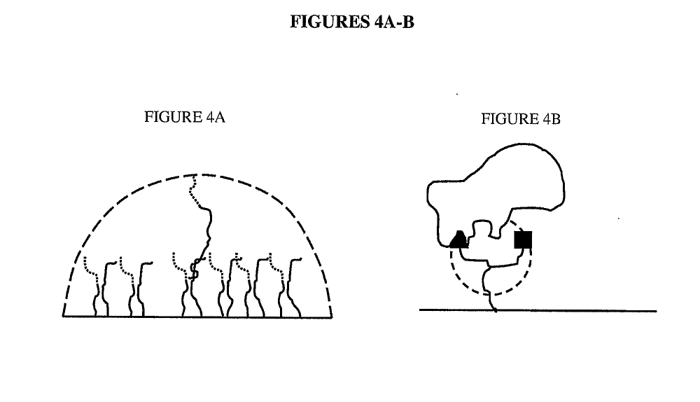

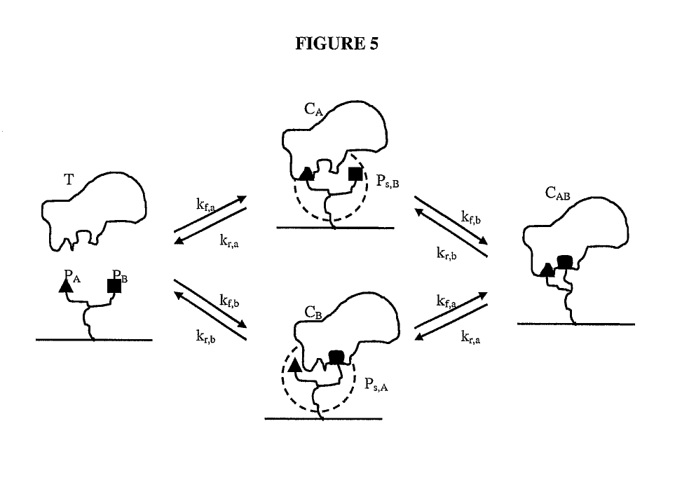

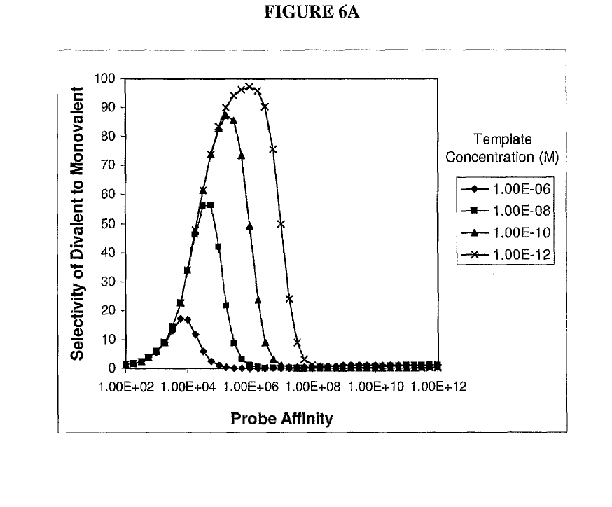

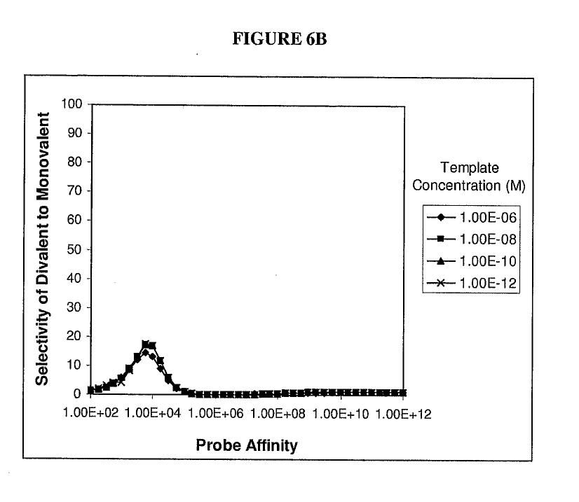

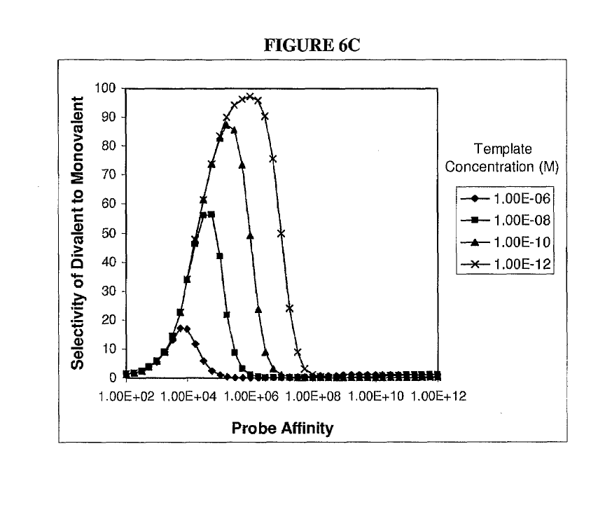

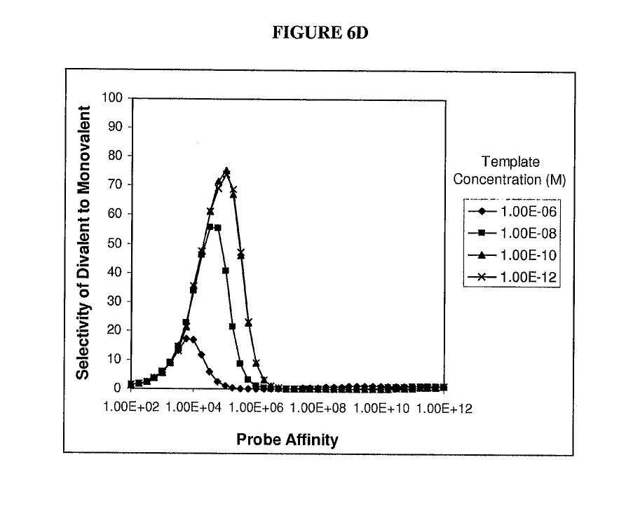

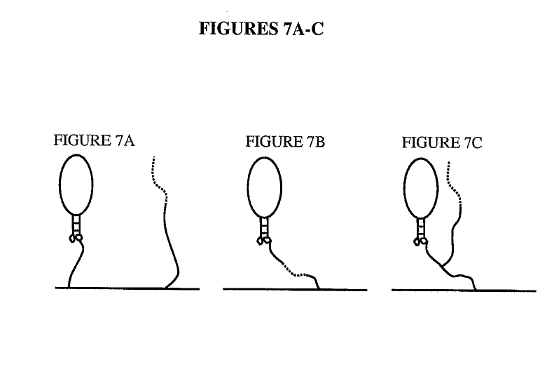

The foregoing equations can be solved simultaneously using numerical methods; however, a simplified representation is ideal for the type of assays that are described herein. Therefore, these equations were applied to develop an effective multivalent equilibrium constant defined as the sum of all products over the reagents as described by Kitov, et al. (Kitov et al., (2003) J. Am. Chem. Soc., 125(52):16271-16284). This algorithm is described for the first time herein for use in developing cooperative binding assays, such as tentacle probe-based assays, cooperative probe assays, and other assays involving other cooperative interactions, including but not limited to drug construct design, cell targeting applications, synthetic antibodies, and the like.

$${Keff}_{di} ≡ \frac{{C}_{A} + {C}_{B} + {C}_{AB}}{P \cdot T} = {Keq}_{A} + {Keq}_{B} + {Keq}_{AB} ; \qquad ( 8 )$$

where equilibrium constants representing the contribution from each complex are derived for microarray or other surfaces where a single analyte due to the distance between binding regions may bind to multiple probes on the array as shown in FIG. 4a:

$$\begin{matrix}{Keq}_{A} = \frac{{C}_{A}}{P \cdot T} = \frac{{k}_{f , A}}{{k}_{r , A}} & ( 9 ) \\ {Keq}_{B} = \frac{{C}_{B}}{P \cdot T} = \frac{{k}_{f , B}}{{k}_{r , B}} & ( 10 ) \\ {Keq}_{AB} = \frac{{C}_{AB}}{P \cdot T} = \frac{{V}_{b}}{{V}_{s}} ( \frac{{P}_{0}}{2} ) {Keq}_{A} {Keq}_{B} & ( 11 )\end{matrix}$$

where P₀ is the total initial probe concentration (P_{A,0}+P_{B,0}). For P_{A,0}=P_{B,0}, P is an averaged probe concentration as follows:

$$P = \frac{{P}_{0}}{2} - \frac{\begin{matrix}\begin{matrix}{C}_{A} ( {Keq}_{A} + {Keq}_{AB} ) + {C}_{B} ( {Keq}_{B} + {Keq}_{AB} ) + \\ {C}_{AB} ( {Keq}_{A} + {Keq}_{B} + 2 {Keq}_{AB} ) +\end{matrix} \\ [ \frac{{C}_{A} {C}_{B} + {C}_{AB} ( {C}_{A} + {C}_{B} + {C}_{AB} )}{{P}_{0} / 2} ] {Keq}_{AB}\end{matrix}}{{Keq}_{A} + {Keq}_{B} + {Keq}_{AB}}$$

It can be shown with much tedium that the averaged probe concentration reduces to a simple expression for every limiting case resulting in a standard Langmuir isotherm:

$${C}_{total} = \frac{( {P}_{0} / 2 ) \cdot {Keff}_{di} \cdot T}{1 + {Keff}_{di} \cdot T} \qquad ( 13 )$$

with a single exception for homovalent binding (Keq_{A}=Keq_{B}) with low cooperativity (Keq_{AB}<<Keq_{A}+Keq_{B}) where the Langmuir isotherm assumes the form:

$${C}_{total} = \frac{( {P}_{0} / 2 ) \cdot {Keff}_{di} \cdot T}{1 + ( 1 / 2 ) \cdot {Keff}_{di} \cdot T} \qquad ( 13 )$$

The effective equilibrium constant (8) for a target which allows for binding to only one probe as shown in FIG. 4b is as follows:

$$\begin{matrix}{K}_{det} = \frac{{C}_{det}}{PT} & ( 14 ) \\ {K}_{cap} = \frac{{C}_{cap}}{PT} & ( 15 ) \\ {P}_{L} {K}_{det} {K}_{cap} = \frac{{C}_{both}}{PT} & ( 16 ) \\ {K}_{eff} = {K}_{det} + {K}_{cap} + {P}_{L} {K}_{det} {K}_{cap} = \frac{{C}_{cap} + {C}_{det} + {C}_{both}}{PT} & ( 17 )\end{matrix}$$

Where K is the equilibrium constant, with added subscripts, cap, det, both, eff, referring to capture probe, detection probe, both capture and detection probes, and effective respectively, P=Pₒ−C_{cap}−C_{det}−C_{both}, T=Tₒ−C_{cap}−C_{det}−C_{both}, where Pₒ and Tₒ are initial probe and target concentrations respectively, and P_{L}=1 molecule/(volume swept out by linker length * Avogadro's number). Total complex formed is almost identical to (13), Cₜₒₜₐₗ=PₒTₒK_{eff}/(1+TₒK_{eff}).
Effective equilibrium constants for higher order systems can be generated by solving the corresponding sets of differential equations.

Several examples of the usefulness of the foregoing algorithms follow, but do not include all of the possible permutations and utilities of having an effective equilibrium constant. The avidity is useful inasmuch as it allows for ready rational design of paired binding partners, such as probe biosensors. It can be used to discover trends in detection limits and amount of complex formed.

The following equations can apply to tentacle probe, CPA, or linear probe systems. Each is a ratio of either detection limits or Langmuir isotherms allowing comparison of specific to nonspecific binding. Batch reactions differ from constant-flow reactions because the target is replenished in constant-flow and may be assumed constant. Each of the subsequent equations can be used for linear systems and cooperative systems. By comparing changes in each of the factors in binding, the best cooperative system for a given application or purpose may be determined.
a. Ratio of batch detection limits:

$$\frac{{T}_{0 , 1}}{{T}_{0 , 2}} = ( \frac{{Keq}_{2}}{{Keq}_{1}} ) ( \frac{1 + ( {P}_{0} - C ) \cdot {Keq}_{1}}{1 + ( {P}_{0} - C ) \cdot {Keq}_{2}} ) \qquad ( 18 )$$

b. Batch resolving power:

$$\frac{{C}_{1}}{{C}_{2}} = \frac{\begin{matrix}( {P}_{0} + {T}_{0} + \frac{1}{{Keq}_{1}} ) - \\ \sqrt{{( {P}_{0} + {T}_{0} + \frac{1}{{Keq}_{1}} )}^{2} - 4 \cdot {P}_{0} \cdot {T}_{0}}\end{matrix}}{\begin{matrix}( {P}_{0} + {T}_{0} + \frac{1}{{Keq}_{2}} ) - \\ \sqrt{{( {P}_{0} + {T}_{0} + \frac{1}{{Keq}_{2}} )}^{2} - 4 \cdot {P}_{0} \cdot {T}_{0}}\end{matrix}} \qquad ( 19 )$$

c. Ratio of constant flow detection limits:

$$\frac{{T}_{0 , 1}}{{T}_{0 , 2}} = \frac{{Keq}_{2}}{{Keq}_{1}} \qquad ( 20 )$$

d. Continuous flow resolving power:

$$\frac{{C}_{1}}{{C}_{2}} = \frac{{Keq}_{2}}{{Keq}_{1}} ( \frac{1 + {Keq}_{1} \cdot {T}_{0}}{1 + {Keq}_{2} \cdot {T}_{0}} ) \qquad ( 21 )$$

The resolving power ratio used for generating model results utilizes continuous flow and homovalent bispecific probe affinities and compares the resolving power equation (17) of CPA to linear probe systems. This measure effectively demonstrates the ability to increase specificity and sensitivity without a tradeoff:

$$\frac{{C}_{di , s}}{{C}_{di , ns}} / \frac{{C}_{s}}{{C}_{ns}} = \frac{\frac{{Keff}_{s}}{{Keff}_{ns}} ( \frac{1 + {Keff}_{ns} \cdot {T}_{0}}{1 + {Keff}_{s} \cdot {T}_{0}} )}{\frac{{Keq}_{s}}{{Keq}_{ns}} ( \frac{1 + {Keq}_{ns} \cdot {T}_{0}}{1 + {Keq}_{s} \cdot {T}_{0}} )} \qquad ( 22 )$$

Other uses for the foregoing models include using models (1)-(4) to look at the rate of binding of the cooperative assay over the rate of binding for the individual components. For example, these models were used to predict that binding rates to extremely strong hairpins that would ordinarily not open in the presence of the target analyte could be enhanced to nearly the binding rates of linear DNA when combined with a capture probe. These predictions with others were used to develop Tentacle Probes.

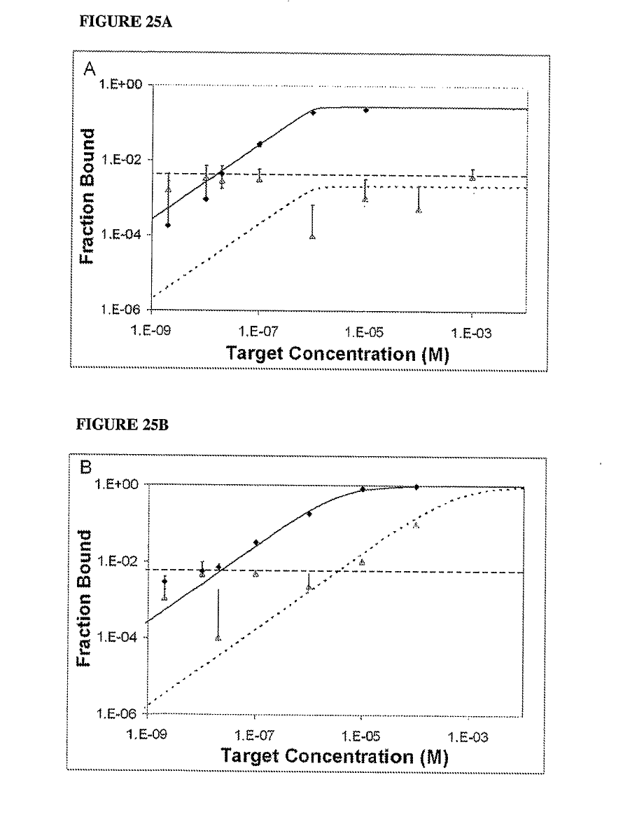

For specialized circumstances where detection of binding can be limited to binding to specific probes within the cooperative set (such as with a Tentacle Probe), the following use applies. Equations (14)-(17) were used to predict trends shown in FIG. 25a allowing for extremely specific detection and were instrumental in the design of Tentacle Probes: Equation (17) can be used to determine the total amount of binding and when multiplied by the ratio of the individual equilibrium constants over the effective equilibrium constant can be used to determine the fraction of binding attributable to each probe component.

Once ideal equilibrium constants and local probe concentrations are determined, probe design can be centered around creating probe lengths with corresponding reaction temperatures that will yield the appropriate estimated equilibrium constant. Linker length is used to control the local probe concentration in equation (17). In an exemplary construction of tentacle probes, a linker length of approximately 3 nm was used (nonaethylene glycol) with individual probes that were designed to have melting temperatures approximately 5° C. under the reaction temperature and a hairpin that was designed to have a melting temperature 30° C. over the reaction temperature. These attributes gave predictions that were desirable for equilibrium constants.

Linking theoretical predictions with practice requires a little calibration. Often the model slightly under predicts. However, by generating melting curves, the correct operating temperature can be quickly determined.

Justification of the use of continuous flow and homovalent systems results from careful analysis of the foregoing equations and reveals that Keff_{di} has the greatest effect for bispecific probe affinities that are nearly equivalent in value. Continuous flow sensors have a greater resolving power. Therefore, for these reasons and due to the increased simplicity of the models under these conditions, in one embodiment of the present invention, a homovalent, continuous flow version of the model is used.

For a homovalent pair, the effective equilibrium constant reduces to:

Keff_{di}=Keq(2+(V_{b}/Vₛ)·(P₀/2)·Keq) (19)

which is identical to the antibody theory as originally derived by Crothers and Metzger and as embodied by Kaufman and Jain (Crothers et al. (1972) Immunochemistry, 9(3):341-357; Kaufman et al. (1992) Cancer Res., 52(15):4157-4167). Others have also used this approximation for modeling bivalent interactions and have experimentally confirmed its accuracy (DeLisi (1976) Antigen Antibody Interactions, Berlin, Springer-Verlag; Perelson et al. (1980) J. Math. Biol., 10(3):209-256; Dmitriev et al. (2002) J. Immunol. Methods, 261(1-2):103-118; Dmitriev et al. (2003) J. Immunol. Methods, 280(1-2):183-202). However, as stated previously, the present invention relates to the use of this model to examine the specificity-sensitivity tradeoff in molecular binding reactions, such as biosensor-based assays.

These derivations of the equilibrium constant apply a different approach from that which has already been done for multivalent systems (Crothers and Metzger (1972) Immunochemistry, 9(3):341-357; Kitov and Bundle (2003) J. Am. Chem. Soc., 125(52):16271-16284; Huskens et al.(2004) J. Am. Chem. Soc., 126(21):6784-6797). However, the result confirms thermodynamic estimates where the avidity is equal to the sum of the free energies of each individual reaction plus an interaction effect, which is typically an entropic penalty (Jencks (1981) Proc. Natl. Acad. Sci. U.S.A., 78(7):4046-4050; Christensen et al. (2003) J. Am. Chem. Soc., 125(24): 7357-7366). The derived equilibrium constant is intuitive in this fashion as the entropic penalty applied is a function of linker length. For relatively large distances between probes, the avidity approaches a separate interaction with no cooperativity between probes, whereas for small distances, the avidity of the multivalent probe approaches a cooperative effect equal to a single molecule with no entropic penalty.

In certain embodiments of the present invention, a cooperative probe for detecting an analyte will comprise a probe set of two or more probes that are attached together and that can specifically bind to different regions of the same target analyte and will have one of or any combination of the following characteristics: (i) an observed melting peak temperature that varies no more than about 10%, 8%, or even 5% with increasing concentration of the analyte when the concentration of analyte is greater than the concentration of the cooperative probe; (ii) a forward rate constant of a probe within the probe set that is greater than 1.5, 2, 3, 4, 5, 8, 10, 15, 20, 50, 100, 300 or 1000 times its noncooperative forward rate constant value, (iii) an analyte binding affinity that is greater than 1.5, 2, 3, 4, 5, 8, 10, 15, 20, 50, 100, 300 or 1000 times the sum of the noncooperative target analyte binding affinities of the individual probes for the target analyte, and (iv) one or more of the probes will not detectably bind to the analyte without the analyte binding to at least one of the other probes.

In certain embodiments, the cooperative probe will be detecting the presence or absence of an analyte while inhibiting non-specific detection of a variant of said analyte comprising an insertion sequence and will comprise a probe set of two or more probes wherein at least one of said probes is specific for said analyte and one of said probes is specific for said variant and having an observed melting peak temperature that varies no more than about 10%, no more than 8% or even no more than 5% with increasing concentration of the variant analyte when the concentration of variant analyte is greater than the concentration of the cooperative probe.

The following specificity, affinity, and kinetics tests can be used to determine whether a probe acts in a cooperative manner in embodiments wherein the set of probes have binding specificity for the same target analyte. The following melting curve test can be used to determine whether a probe acts in a cooperative manner in embodiments wherein the set of probes have binding specificity for the same target analyte or wherein cooperativity is used to refer to enhanced characteristics contributing to the inhibition of identification of an analyte.

In certain embodiments, if one or more of the probes will not detectably bind to the analyte without the analyte binding to the other probes in the cooperative system than it can be deemed cooperative. The individual probes in the probe set comprising the cooperative probe can be tested individually for binding and detection. If none of the probes bind when tested individually, or if none of the detection probes bind in the case of cooperative assays that only record the signal from the detection probes by itself, but they do bind when in the cooperative system, using the same buffers, reaction temperatures and instrumentation, than the probe can be deemed to have to have cooperative characteristics for specificity.

The affinities of the individual components of the cooperative assay can be tested in addition to the affinity of the cooperative probe. Methods of determining the affinity include using fluorescent or radio labels and measuring the forward and reverse rate constants, in which the affinity is the forward rate constant divided by the reverse rate constant. Another method is to label the analyte and to perform titrations over several orders of magnitude on a microarray or similar device that has a very low probe concentration (e.g. more than 10× lower than the analyte concentrations used in the titrations). The concentration of analyte at which half the probes are bound (e.g. half maximal fluorescence) is equal to the dissociation constant, or the inverse of the affinity. If the cooperative avidity (effective affinity) is more than the sum of the individual equilibrium constants, using the same buffers, reaction temperatures and instrumentation, then the probe set can be considered cooperative in affinity. Typically, it will be more than 1.5 times the sum of the individual equilibrium constants.

The kinetics of the individual components of the cooperative assay can be tested in addition to the kinetics of the cooperative probe. Kinetic forward rate constants are determined for the slowest component in the cooperative set in addition to the effective rate constant of binding. If it is not immediately apparent which is the slowest probe in the set, then all probes can be tested and the probe possessing the lowest rate constant can be deemed the slowest. If the effective forward rate constant for cooperative binding is more than the value of the slowest binder using the same buffers, reaction temperatures and instrumentation, then the probe set can be deemed cooperative in kinetics. Typically, the effective forward rate constant for cooperative binding will be more than 1.5 times the value of the slowest binder using the same buffers. Common methods of measuring rate constants include observing changes in fluorescence (such as with hairpins) over time or using radiolabeled techniques or with Surface Plasmon Resonance in the presence of an excess of target (e.g. more than 10 fold). The data is typically fit to the following equation to determine the forward rate constant:

F=Fₘₐₓ(1−e^{(-k}^{_{f}}^{T)t})

Where F is fluorescence, Fₘₐₓ is the maximum fluorescence achieved at equilibrium, k_{f} is the effective forward rate constant, T is the target concentration and t is time.

A melting curve test can be applied to a probe set that has one or more detection probes that are monitored in an assay as opposed to the whole set. Melting curves of the probe set are generated for the analyte on a Stragene Mx4000 plate reader or comparable product that is capable of observing the relative binding of the detection probes. All probes and target sequences are suspended in the recommended reaction buffers or are suspended in TE buffer (10 mM Tris-Cl, 1 mM EDTA, pH 7.0) with 0.18 M NaCl and 0.1% SDS. 20 μL of solutions with final probe concentration of 50 nM and target concentrations of 50 nM, 500 nM, 5 μM and 50 μM are prepared. Signal is monitored from 90° C. to 15° C. with a fifteen minute incubation period in between each 1° C. increment. The experiment is also repeated from 15° C. to 90° C. An example of this type of analysis is shown in FIG. 24a& b. Melting peaks are calculated by subtracting the fluorescence at each temperature from the fluorescence at the temperature preceding it. The peak is the highest fluorescent change from one temperature to the next and corresponds to the temperature at which approximately half the bound probes have melted. A probe set that possesses melting peaks for the given experiments that shift less than 10% from the highest value or in ideal cases, less than 5%, is considered to possess immobile melting curves.

Melting peaks typically approximate the temperature at which half of the template is bound. However in Tentacle Probes, there is a difference between the actual melting peak and the observed melting peak. Since Tentacle Probes possess at least one capture probe in addition to a detection probe, there is the possibility of binding analyte via the capture probe that is not detected. Thus, the observed melting peak reflects only the binding of analyte to the detection probe(s). This is in contrast to the actual melting peak which represents the temperature at which approximately half of the analyte is melted off both capture and detection probes. For purposes of this application, all references to the melting peak refer only to the observed melting peak, or the temperature at which approximately half the analyte has melted off the detection probe(s).

### Analyte

The present invention provides a method for detecting an analyte or a plurality of analytes by using tentacle probes. The present invention can be used to analyze both biological and non-biological analytes. Suitable biological analytes include, but are not limited to, proteins, peptides, nucleic acid sequences, peptide nucleic acids, antibodies, antigens, receptors, molecules, biological cells, microorganisms, cellular organelles, cell membrane fragments, bacteriophage, bacteriophage fragments, whole viruses, viral fragments, and small molecules such as lipids, carbohydrates, amino acids, drug substances, and molecules for biological screening and testing. An analyte can also refer to a complex of two or more molecules, for example, a ribosome with both RNA and protein elements or an enzyme with substrate attached.

In general, the analyte of the present invention is one that is able to specifically bind to at least a portion of the detection probe. The phrase “specifically bind(s)” or “bind(s) specifically” when referring to a detection probe refers to a detection probe that has intermediate or high binding affinity, exclusively or predominately, to a target molecule. The phrase “specifically binds to” refers to a binding reaction which is determinative of the presence of a target in the presence of a heterogeneous population of other biologics. Thus, under designated assay conditions, the specified binding region bind preferentially to a particular target and do not bind in a significant amount to other components present in a test sample. Specific binding to a target under such conditions can require a binding moiety that is selected for its specificity for a particular target. A variety of assay formats can be used to select binding regions that are specifically reactive with a particular analyte. Typically a specific or selective reaction will be at least twice background signal or noise and more typically more than 10 times background.

In certain embodiments wherein the detection probe is a nucleic acid, the analyte can be, for example, anti-oligonucleotide antibodies, polynucleotide binding proteins, or complementary nucleic acid fragments (including DNA sequences, RNA sequences, and peptidyl nucleic acid sequences). Generally, upon specific binding interactions with a target analyte, the tentacle probe changes its conformation from a closed stem-loop or hairpin form into an open form, resulting in the separation of the first and second signal altering moiety and thus generating a change in detectable signals.

Sources of analytes can be isolated from organisms and pathogens such as viruses and bacteria or from an individual or individuals, including, but not limited to, skin, plasma, serum, spinal fluid, lymph fluid, synovial fluid, urine, tears, blood cells, organs, tumors, and also samples of in vitro cell culture constituents, such as conditioned medium resulting from the growth of cells in cell culture medium, recombinant cells and cell components. Analytes can also be from environmental samples such as air or water samples, or may be from forensic samples from biological or non-biological samples, including clothing, tools, publications, letters, furniture, etc. Additionally, analytes can also come from synthetic sources. The analytes in the present invention can be provided in a sample that can be a crude sample, a partially purified or substantially purified sample, or a treated sample, where the sample can contain, for example, other natural components of biological samples, such as proteins, lipids, salts, nucleic acids, and carbohydrates.

In certain exemplary embodiments of the invention, the analyte will be Yersinia pestis or Bacillus anthracis.

### Tentacle Probe

The present invention provides, inter alia, tentacle probes. Tentacle probes can have many different combinations of detection and capture probes. In preferred embodiments, the detection probe is in predominantly an open conformation when bound to said target analyte and is in predominantly a closed conformation when not bound to said target analyte. The change in conformation generates a change in detectable signal. The detection probe comprises a first binding region and the capture probe comprises a second binding region that is different than the first binding region. In other words, the second binding region binds to a region on the target analyte that is distinct and separate from the first target binding region. In exemplary embodiments when the tentacle probes are used for detecting target analyte in a sample, the first and second binding region are specific for the target analyte.

The tentacle probes of the present invention can comprise a first arm region and a second arm region that form a stem duplex when the probe is in a closed conformation and are separated when the probe is in an open conformation. The arm regions can be attached to a signal altering moiety although it is not necessary for detection. In certain embodiments, one of the arm regions is attached to a signal altering moiety. In other embodiments, both of the arm regions are attached to signal altering moieties. The target binding region on the detection probe can be intermediate to said first and second arm region, although it need not be. In certain embodiments, the first or second arm will comprise all or part of the target binding region. In others, the first and second arm will comprise a part of the target binding region.

The capture probe can be attached to the detection probe directly or indirectly. The capture probe is attached to the detection probe in such way that the detection and capture probes can cooperatively interact with the target. The capture probes and detection probes of the present invention are not meant to function as primers. In certain embodiments, the detection and/or capture probe not only will not function as a primers but will be incapable of initiating of initiating nucleic acid replication or amplification. In certain embodiments, this will be because the detection probe and/or capture probe of a tentacle probe will be non-extendable. In certain embodiments, the detection probe and/or capture probe will be blocked to prohibit polymerase catalyzed extension of the probe.

In certain embodiments, the target analyte is single stranded or double stranded nucleic acid and the capture probe and the detection probe comprise a sequence that is complementary to the same stand of nucleic acid and that is present on non-multiplied nucleic acid.

It will be understand that either or both of the capture and detection probe can comprise additional binding regions.

In certain embodiments, the capture probe is non-extendible, i.e., the capture probe cannot act as a primer. In certain embodiments wherein the target analyte is single stranded or double stranded nucleic acid, the capture probe and detection probe can comprise a sequence that is complementary to the same stand of nucleic acid and that is present on non-multiplied nucleic acid.

Although the capture probe can be a probe that always remains in the same conformation whether bound or not bound to a target analyte, it can also, in certain embodiments, look very much like a detection probe. It can be in predominantly an open conformation when bound to a target analyte and in predominantly a closed conformation when not bound to a target analyte. The change in conformation can also, if desired, generate a change in detectable signal. The capture probe can, if desired, comprise a first arm region and a second arm region that form a stem duplex when the probe is in a closed conformation and are separated when the probe is in an open conformation. If desired, the arm regions can be attached to a signal altering moiety. One or both of the arm regions can be attached to a signal altering moiety. The target binding region on the detection probe can be intermediate to said first and second arm region. Alternatively, the first or second arm can comprise all or part of the target binding region or the first and second arm can comprise a part of the target binding region.

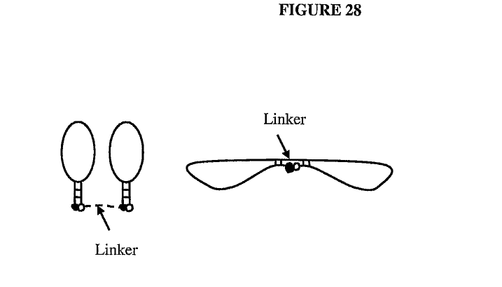

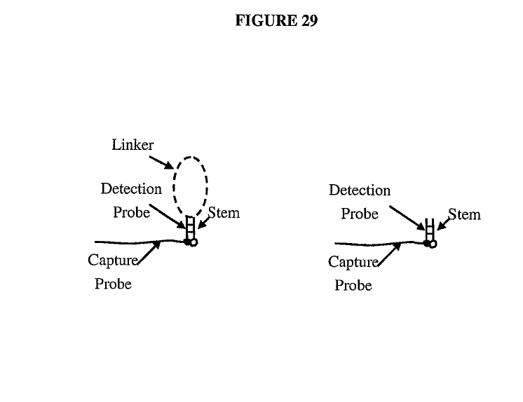

A tentacle probe of the present invention can have a detection probe bound to one signaling altering moiety and a capture probe that looks very much like the detection probe bound to another signaling altering moiety. When presented with the target analyte, both probes can bind the analyte thus generating a change in detectable signal. An example of this embodiment is shown in FIG. 28.

In certain embodiments, the tentacle probes will have different combinations of detection probes and capture probes. For example, the tentacle probe can have two or more detection probes as described herein attached together with one or more capture probes as described herein.

A detection probe can comprise two arm regions that form a stem duplex when in a closed conformation, and an open conformation when the first and second arm regions are separated. The term “duplex” is used in its broadest sense to refer to a stem having two principal elements or parts. In certain embodiments, the arm regions will be complementary to each other and will hybridize to form a Watson-Crick paired stem duplex. In other embodiments, the arm regions will not be complementary but can be brought together to form a stem duplex by other forces, including, for example, electrostatic forces, hydrophobic interactions, magnetic forces, and the like. For example, in certain embodiments, the two arm regions will be brought together by a receptor ligand pair, positively and negatively charged amino acids, a streptavidin and biotin pair, or a hydrophobic fluorophore and quencher pair.

The term “open” as used herein in reference to a probe condition is used to indicate a change in molecular conformation from the “closed” condition. In some embodiments the change in molecular conformation can be a change in secondary structure. For example, a stem loop structure can be “closed” when the stem duplex is formed and “open” when dissociated. In other embodiments the change in conformation can be in tertiary or quaternary structure. For example, two proteins or two strands of nucleic acids when in contact or close proximity with each other would be considered “closed” and upon dissociation would be considered “open.”

The conformation of the detection probe can be readily altered from one to the other by a target analyte, which can bind to the first target binding region. In the absence of a target analyte, the detection probe is predominantly in the closed conformation, in which the two signal altering moieties are in proximity with each other for effective interactions. In the presence of a target analyte capable of binding to the first target binding region, the detection probe is predominantly in the open conformation and separates the two signal altering moieties apart, generating new detectable signals. In certain embodiments wherein the target analyte is nucleic acid, the analyte will have a sequence that is complementary to that of the target binding region.

In certain embodiments, the capture and detection probes can be designed such that the detection probe will not bind unless the capture probe binds first. An exemplary case is where the strength of the hairpin conformation is greater than the strength of the binding interaction between the detection probe and the analyte, causing it to remain shut unless a cooperative interaction takes place by first binding to the capture probe.

Or in the case of multiple probes, they can be designed such that all are required to be an exact match or part are required to be a match in order to produce a binding event. An exemplary case is where the binding affinity of each interaction is too low to produce a sustainable binding event, but the sum of the parts and the cooperative interaction (as exemplified in equation (17)) produces a sustainable binding event.

Tentacle probes of the present invention can also be designed so that when used in a detection system, they reduce the number of false positives. In certain embodiments, they will eliminate or substantially eliminate false positives. Certain of the tentacle probes of the present invention have a detection probe and a capture probe wherein target analyte is nucleic acid and the detection probe and capture probe comprise a sequence specific for different regions on the same target analyte. In certain embodiments, however, the detection probe can comprise a binding region specific for a sequence present on a target nucleic acid but not on a variant of said nucleic acid while the capture probe comprises a binding region specific for a sequence present on the variant. These tentacle probes can be used for detecting a target nucleic acid in a sample while inhibiting detection of a variant of the target nucleic acid comprising an insertion sequence. If using these probes in an amplification reaction, they can also prevent amplification of the variant analyte. Alternatively, the detection probe and capture probe comprise a sequence specific for a sequence present on a target nucleic acid but not on a variant of said nucleic acid while a linker linking the detection and capture probe can comprise a sequence specific for the insertion.

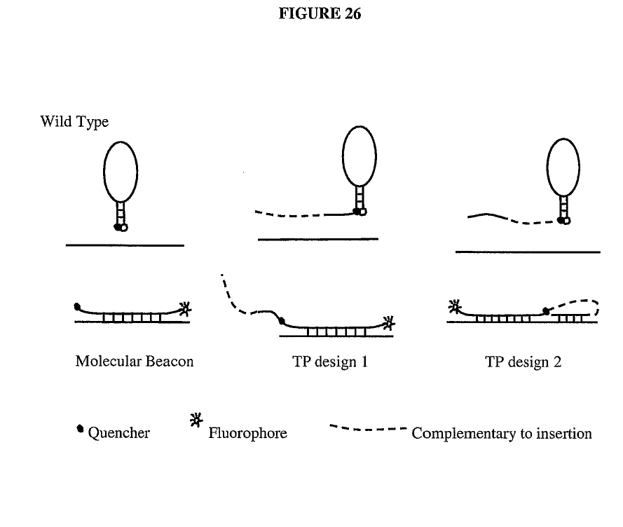

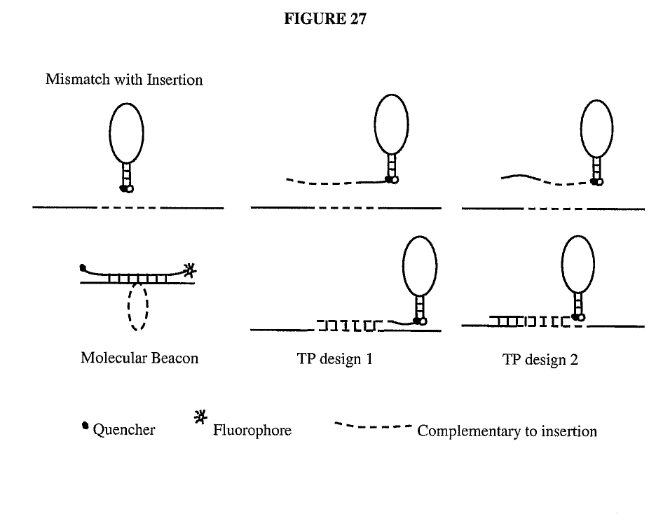

FIGS. 26 and 27 demonstrate two exemplary methods of using tentacle probes to bind to a target analyte and not to a variant of the analyte. In such a system, the tentacle probe can be designed such that the one binding region binds to an insertion sequence on the variant while another binding region binds to a region present on the target. In the presence of the target analyte, the binding region on detection probe binds to the target and the detection probe changes from a closed to an open conformation. The capture probe remains unbound. In the presence of the variant, the capture probe binds to the insertion, thereby preventing the insertion from evading detection, and the detection probe does not bind to the variant sequence. Alternatively, the tentacle probe can be designed such that the first and second binding region binds to the insertion sequence while a linker linking the capture and detection probe binds to the insertion. In the presence of the target analyte, the first and second binding region bind to the target, the linker remains unbound, and the detection probe changes from a closed to an open confirmation. In the presence of the variant, the linker binds to the insertion, thereby preventing the insertion from evading detection, and the detection probe does not bind to the variant sequence.

For optimal cooperative interactions with the intended target analyte, the detection probe and the capture probes are in close proximity in space to each other. The suitable distance between capture and detection probes depends on the size of the analyte and the strength of the affinity interactions. Equation (17) can be used to determine the preferred distance between capture and detection probe.

Depending on the size of the analyte and strength of the affinity interactions, the suitable distance between the detection probe and at least one of the capture probes is generally no greater than 1000 nm, no greater than 500 nm, or no greater than about 100 nm for a large analyte such as a cell, and no greater than about 90 nm, no greater than about 80 nm, no greater than about 50 nm, no greater than about 40 nm, no greater than about 30 nm, no greater than about 20 nm, no greater than about 15 nm, or no greater than about 10 nm for a smaller analyte. In certain embodiments, the linker will be less than 10 nm, less than 9 nm, or even less than 5 nm.

In certain preferred embodiments, the tentacle probes of the present invention have any combination of the following characteristics (i) an observed melting peak temperature that varies no more than about 10% with increasing concentration of the analyte when the concentration of analyte is greater than the concentration of the tentacle probe; (ii) a forward rate constant of the capture probe or detection probe that is greater than one and a half times its noncooperative forward rate constant value (iii) an analyte binding affinity that is greater than one and a half times the sum of the noncooperative target analyte binding affinities of the individual probes for the target analyte; and (iv) at least one of the probes will not detectably bind to the analyte without the analyte binding to at least one of the other probes.

In certain exemplary embodiments, the detection and capture probes are attached to a common surface in close proximity in space to each other so that the target analyte is able to interact with both the first and second target binding region simultaneously and cooperatively. The detection and capture probes can be attached to various surfaces, including the surface of a solid support, a nanotube, a cell, or a microorganism such as a bacterium, virus, or phage. Suitable solid supports include, but are not limited to cyclo olefin polymers and copolymers, acrylamide, cellulose, nitrocellulose, glass, polystyrene, polyethylene vinyl acetate, polypropylene, polymethacrylate, polyethylene, polysilicates, polyethylene oxide, polycarbonates, teflon, fluorocarbons, nylon, silicon rubber, collagen, polyanhydrides, polyglycolic acid, polylactic acid, polyorthoesters, polypropylfumarate, glycosaminoglycans, and polyamino acids. A solid support or matrix can be in one of the many useful forms including thin films or membranes, plates such as various formats of microtiter plates, beads such as magnetic beads or latex beads, bottles, dishes, fibers, woven fibers, shaped polymers, particles, microarrays, microfluidic channels, microchips, microparticles such as microspheres, and nanoparticles. Methods of attaching the capture and detection and capture probes to a surface are known in the art and include, without limitation, direct adhesion to the surface such as plastic, use of a capture agent, chemical coupling, and via a binding pair such as biotin-avidin. The detection and capture probes can independently have a tether to facilitate the attachments to the surface signals.

A. Detection and Capture Probe

Exemplary tentacle probes of the present invention are probes that comprise a detection probe and a capture probe that act cooperatively to identify or inhibit identification of an analyte. The detection probe can form a hairpin conformation by itself or with the addition of other nucleic acids, i.e., it can exist in an open or closed conformation depending on whether it is bound to a target analyte. In certain embodiments of the present invention, binding of the capture probe to a target analyte is required in order for binding of the detection probe to the analyte and subsequent detection of the analyte.

In some embodiments, the capture probe can also form a hairpin by itself or with the addition of other nucleic acids, and can, in certain embodiments, generate its own change in detectable signal depending on whether it is in a open or closed conformation, irrespective of the detection probe. In these embodiments, a combined signal from the detection and capture probe can be used to detect the presence or absence of a target analyte. In other embodiments, the capture probe exists only in an open, e.g., linear conformation and cannot form a hairpin structure.

In certain embodiments, the detection and capture probe are all oligonucleotides, although the need not be oligonucleotides. Each oligonucleotide probe can comprise, for example, naturally occurring nucleotide residues, modified nucleotide residues, or combinations thereof. Exemplary nucleic acids include, but are not limited to, conventional ribonucleic acid (RNA), deoxyribonucleic acid (DNA), and chemical analogs of these molecules such as a locked nucleotide analog (“LNA”) and a peptide nucleic acid (“PNA”).

A vast variety of modified nucleic acid analogs can also be used in the present invention, including backbone modifications, sugar modifications, nitrogenous base modifications, or combinations thereof. The “backbone” of a natural nucleic acid is made up of one or more sugar-phosphodiester linkages. The backbone of a nucleic acid of the present invention can also be made up of a variety of other linkages known in the art, including peptide bonds, also known as a peptide nucleic acid (Hyldig-Nielsen et al., PCT No. WO 95/32305; Egholm (1992) J. Am. Chem. Soc. 114:1895; Meier et al. (1992) Chem. Int. Ed. Engl. 31:1008; Nielsen (1993) Nature 365:566; Carlsson et al. (1996) Nature 380:207); phosphorothioate linkages (Mag et al. (1991) Nucleic Acids Res. 19:1437; U.S. Pat. Nos. 5,644,048; 5,539,082; 5,773,571; 5,977,296, and 6,962,906); phosphorodithioate linkages (Briu et al. (1989) J. Am. Chem. Soc. 111:2321); phosphoramidate linkages (Beaucage et al. (1993) Tetrahedron 49(10):1925; Letsinger (1970) J. Org. Chem. 35:3800; Sprinzl et al. (1977) Eur. J. Biochem. 81:579; Letsinger et al. (1986) Nucleic Acids Res. 14:3487; Sawai et al. (1984) Chem. Lett. 805; Letsinger et al. (1988) J. Am. Chem. Soc. 110:4470; and Pauwels et al. (1986) Chemica Scripta 26:1419); methylphosphonate linkages; O-methylphosphoroamidite linkages (Eckstein, Oligonucleotides and Analogues: A Practical Approach, Oxford University Press); or combinations thereof.

Other suitable linkages include positive backbones (Denpcy et al. (1995) Proc. Natl. Acad. Sci. USA 92:6097); non-ionic backbones (U.S. Pat. Nos. 5,386,023, 5,637,684, 5,602,240, 5,216,141 and 4,469,863; Kiedrowski et al. (1991) Angew. Chem. Intl. Ed. English 30:423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research,” Ed. Y. S. Sanghui and P. Dan Cook; Mesmaeker et al. (1994), Bioorganic & Medicinal Chem. Lett. 4:395; Jeffs et al. (1994) J. Biomolecular NMR 34:17; Horn et al. (1996) Tetrahedron Lett. 37:743), and non-ribose backbones (U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook).

Sugar moieties of a nucleic acid can be either ribose, deoxyribose, or similar compounds having known substitutions, such as, for example, 2′-O-methyl ribose, 2′-halide ribose substitutions (e.g., 2′-F), and carbocyclic sugars (Jenkins et al. (1995), Chem. Soc. Rev. pp 169-176). The nitrogenous bases are conventional bases (A, G, C, T, U), known analogs thereof, such as inosine (I) (The Biochemistry of the Nucleic Acids 5-36, Adams et al., ed., 11th, 1992), known derivatives of purine or pyrimidine bases, such as N⁴-methyl deoxyguanosine, deaza- or aza-purines, deaza- or aza-pyrimidines, pyrimidine bases having substituent groups at the 5 or 6 position, purine bases having an altered or a replacement substituent at the 2, 6 or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines (Cook, PCT No. WO 93/13121) and “abasic” residues where the backbone includes no nitrogenous base for one or more residues of the polymer (Arnold et al., U.S. Pat. No. 5,585,481).

Each oligonucleotide probe of the tentacle probe in accordance with the present invention can vary from about five nucleotides in length to over 1,000 nucleotides in length.

In some embodiments of the cooperative probe assay, the probes are designed for maximum specificity. As such, the individual probe affinities are preferably between about 10² M⁻¹ and about 10⁸ M⁻¹, more preferably between about 10² M⁻¹ and about 10⁶ M⁻¹, and even more preferably between about 10⁴ M⁻¹ and about 10⁶ M⁻¹. For cooperative probe assays that operate based on base-pairing between nucleotides, preferred probe lengths to achieve these affinities range between about 5 and about 25 bases, more preferably between about 10 and about 25 bases, and even more preferably between about 15 and about 25 bases.

In some embodiments of the cooperative probe assay, large affinities are desired for maximum sensitivity or to allow binding of variants. In this case, homovalent probe affinities that comprise the cooperative probe preferably range between about 10⁶ M⁻¹ and about 10¹⁰⁰ M⁻¹, more preferably between about 10⁶ M⁻¹ and about 10⁵⁰ M⁻¹, and even more preferably between about 10⁸ M⁻¹ and about 10⁵⁰ M⁻¹. For embodiments targeting nucleic acids, some probe lengths range preferably between about 20 and about 70 nucleotides, more preferably between about 20 and about 50 nucleotides, and most preferably between about 20 and about 40 nucleotides.

In some embodiments of the Tentacle Probe, capture and detection probes have the same affinities for the target without the presence of a hairpin conformation in the detection probe. For specific interactions, these affinities preferably range between about 10² M⁻¹ and about 10⁸ M⁻¹, more preferably between about 10² M⁻¹ and about 10⁶ M⁻¹, and even more preferably between about 10⁴ M⁻¹ and about 10⁶ M⁻¹. For cooperative probe assays that operate based on base-pairing between nucleotides, probe lengths to achieve these affinities preferably range between about 5 and about 25 bases, more preferably between about 10 and about 25 bases, and even more preferably between about 15 and about 25 bases.

In some embodiments of the present invention, capture and detection probes have the same affinities for the target without the presence of a hairpin conformation in the detection probe. For mutation tolerance or high affinity interactions, affinities that comprise the Tentacle Probe preferably range between about 10⁶ M⁻¹ and about 10100 M⁻¹, more preferably between about 10⁶ M⁻¹ and about 10⁵⁰ M⁻¹, and even more preferably between about 10⁸ M⁻¹ and about 10⁵⁰ M⁻¹. For embodiments targeting nucleic acids, some probe lengths range between about 20 and about 70 nucleotides, between about 20 and about 50 nucleotides, between about 20 and about 40 nucleotides.

In some embodiments, the Tentacle Probe possesses a hairpin structure where two arms with affinity for each other close the hairpin. For specific interactions that require the capture probe to bind first in order for a detection to occur, the hairpin melting temperature ranges preferably between about 20° C. and about 75° C. above the reaction temperature, more preferably between about 20° C. and about 50° C. above the reaction temperature, and even more preferably between about 25° C. and about 40° C. above the reaction temperature. The corresponding stem lengths are preferably between about 3 and about 30 base pairs, more preferably between about 6 and about 20 base pairs, and even more preferably between about 8 and about 15 base pairs.

In some embodiments, a high stem G-C content is used to have higher stem melting temperatures for shorter sequences. In some embodiments, the stem is made in part or entirely complementary to the detection probe sequence such that the two stems are forced apart from each other and there is no chance of hybridization occurring without separating the stems.

In some embodiments, the Tentacle Probe possesses a hairpin structure where two arms with affinity for each other close the hairpin, but where it is not important for the capture probe to bind first in order for detection to occur. In these embodiments, the hairpin melting temperature ranges preferably between about 5° C. and about 30° C. above the reaction temperature, more preferably between about 10° C. and about 30° C. above the reaction temperature, and even more preferably between about 15° C. and about 25° C. above the reaction temperature. The corresponding stem lengths are preferably between about 3 and about 15 base pairs, more preferably between about 5 and about 15 base pairs, and even more preferably between about 5 and about 10 base pairs.

The selection of the lengths for the capture and detection probes are discussed in details herein in the section of “Design of Tentacle Probes.” The probes can be single-stranded or double stranded nucleic acid. The detection probe typically forms a hairpin by itself or with the addition of other nucleic acids, whereas the capture probe can be linear, branched, circular, or combinations thereof. The capture probe can also form a hairpin by itself.

Typically, each capture probe contains only one binding region. In certain embodiments, the capture probe can contain two or more binding regions that bind to distinct parts of a molecule. In certain embodiments, the capture probe can contain two or more binding regions that each bind to distinct parts of the target analyte, which are connected together via one or more linkers.

If desired, the oligonucleotide probe can be rendered non-extendable in that additional nucleotide cannot be added to the probe. The oligonucleotide probe can be rendered non-extendable, for example, by modifying the 3′ end of the probe such that the hydroxyl group is no longer capable of participating in elongation. The hydroxyl group of a 3′ natural occurring nucleotide simply can be modified with a variety of functional groups. For example, the 3′ end of the capture probe can be blocked with a linker, which is used to attach the capture probe to the detection probe. Alternatively, the oligonucleotide probe can be rendered non-extendable by incorporating a nucleotide analog that lacks a 3′ hydroxyl group or can not function as a substrate of a polymerase for extension. In the present invention, the detection and capture probes may each independently be non-extendable.

Accordingly in the methods of the present invention, “blocking” can be achieved in many different ways, for example, by using non-complementary bases or by adding a chemical moiety such as biotin or a phosphate group to the 3′ hydroxyl of the last nucleotide or by removing the 3′-OH or by using a nucleotide that lacks a 3′-OH such as a dideoxynucleotide. A blocking moiety, in certain instances will serve a dual purpose by also acting as a label for subsequent detection.

The oligonucleotide probes of the tentacle probe can be made using various methods known in the art. The nucleic acid can be, for example, a recombinant polynucleotide, a natural polynucleotide, or a synthetic or semi-synthetic polynucleotide, or combinations thereof. Various functional groups such as, for example, amino, carbonyl, carboxylic acid, halide, hydrazine, carbohydrazide, thiol groups can also be incorporated into any position of the nucleic acid as long as the functional group does not affect the specificity and binding affinity, and the effective interactions between the two signal altering moieties when in closed conformation. The functional groups are useful for incorporation of labels (signal altering moieties) and conjugation of two or more oligonucleotide segments, such as the detection probe and the capture probe.

B. Signal Altering Moieties

In addition to the binding region, the detection probe and capture probe can contain signal altering moieties. In certain embodiments, the capture probe will not contain a signal altering moiety, i.e., the signal altering moieties will be present on the detection probe only. In certain embodiments, the detection probe will contain arm regions with the first arm attached to a first signal altering moiety and the second arm region attached to a second signal altering moiety that is different than the first signal altering moiety. In the absence of a target, the detection probe exists predominantly in a closed conformation, a stem-loop or hairpin, with the two arms forming a stem duplex, and thus bringing the two signal altering moieties in proximity for effective interaction, including, but not limited to, interaction between a molecular energy transfer pair or enzyme-inhibitor pair. In some instances only one signal altering moiety will be required. For example, fluorescence polarization which produces changes in signal for hybridized versus unhybridized DNA. Fluorescence polarization is described, for example, in U.S. Pat. No. 5,445,935, incorporated herein by reference in its entirety. In some cases, no signal altering moiety will be required such as in electrochemistry where the current changes based on the presence or absence of a hairpin.

In general, upon binding to a target analyte, the interactions between the detection probe and the target analyte shifts the equilibrium predominantly towards an open conformation. In this open conformation, the two arms are separated from each other, thus generating a change in detectable signals that can be used to detect or quantitate the target analyte. It will be understood that the arms can be attached to multiple signal altering moieties if so desired.

In certain alternative embodiments, the tentacle probe will comprise a detection probe and a capture probe also containing two arm regions that can be bound together. Each probe can have one fluorophore attached to one of the two arm regions. Each probe will bind to a different region on the target analyte. In the absent of a target, the detection probe exists predominantly in a closed conformation, a stem-loop or hairpin, with the two arms forming a stem duplex, and thus bringing the two signal altering moieties in proximity for effective molecular energy transfer. Upon binding to a target analyte, the interactions between the detection probe and the target analyte shifts the equilibrium predominantly towards to an open conformation. In this open conformation, the two arms are separately from each other and prevents substantial molecular energy transfer between the two signal altering moieties, thus generating a change in detectable signals that can be used to detect or quantitate the target analyte.

A variety of signal altering groups are suitable for use in the tentacle probes of the present invention. For example, signal altering moieties can include a wide range of energy donor and acceptor molecules to construct resonance energy transfer probes. Energy transfer can occur, for example, through fluorescence resonance energy transfer, bioluminescence energy transfer, or direct energy transfer. Fluorescence resonance energy transfer occurs when part of the energy of an excited donor is transferred to an acceptor fluorophore which re-emits light at another wavelength or, alternatively, to a quencher group that typically emits the energy as heat. There is a great deal of practical guidance available in the literature for selecting appropriate donor-acceptor pairs for particular probes, as exemplified by the following references: Pesce et al., Eds., Fluorescence Spectroscopy (Marcel Dekker, New York, 1971); White et al., Fluorescence Analysis: A Practical Approach (Marcel Dekker, New York, 1970); and the like. The literature also includes references providing exhaustive lists of fluorescent and chromogenic molecules and their relevant optical properties, for choosing reporter-quencher pairs (see, for example, Berlman, Handbook of Fluorescence Spectra of Aromatic Molecules, 2nd Edition (Academic Press, New York, 1971); Griffiths, Colour and Constitution of Organic Molecules (Academic Press, New York, 1976); Bishop, Ed., Indicators (Pergamon Press, Oxford, 1972); Haugland, Handbook of Fluorescent Probes and Research Chemicals (Molecular Probes, Eugene, 1992) Pringsheim, Fluorescence and Phosphorescence (Interscience Publishers, New York, 1949); and the like. Further, there is extensive guidance in the literature for derivatizing acceptor and quencher molecules for covalent attachment via readily available reactive groups that can be added to a molecule. Many donor and acceptor molecules, in addition to synthesis techniques are also readily available from many synthesis companies, such as Biosearch Technologies. All of the above publications are herein incorporated by reference in their entirety.

In certain embodiments of the present invention, the first signal altering moiety is a fluorophore and the second signal altering moiety is a fluorescence quencher. In the absence of a target analyte, the tentacle probe is predominately in a closed conformation. Thus, the two signal altering moieties are close enough in space for effective molecular energy transfer and the fluorescent signal of the fluorophore is essentially completely suppressed by the fluorescence quencher. In the present of a target analyte, the interactions between the target analyte and the tentacle probe change the conformation of the detection probe into an open state. Thus, the two signal altering moieties are far apart from each in space and the fluorescent signal of the fluorophore is restored for detection.

In certain alternative embodiment, the first signal altering moiety and the second signal altering moieties are both fluorophores that emit a certain wavelength when in close proximity and another when further apart.

Suitable fluorophores include, but are not limited to, coumarin, fluorescein (e.g., 5-carboxyfluorescein (5-FAM), 6-carboxyfluorescein (6-FAM), 2′,4′,1,4,-tetrachlorofluorescein (TET), 2′,4′,5′,7′,1,4-hexachlorofluorescein (HEX), and 2′,7′-dimethoxy-4′,5′-dichloro-6-carboxyfluorescein (JOE)), Lucifer yellow, rhodamine (e.g., tetramethyl-6-carboxyrhodamine (TAMRA), and tetrapropano-6-carboxyrhodamine (ROX)), 4,4-difluoro-5,7-dimethyl-4-bora-3a,4a-diaza-s-indacene (BODIPY), DABSYL, DABCYL, cyanine (e.g., Cy3, Cy5, and Cy7), cosine, Texas red, ROX, quantum dots, anthraquinone, nitrothiazole, and nitroimidazole compounds, Quasar and Cal-fluor dyes, and dansyl derivatives. Combination fluorophores such as fluorescein-rhodamine dimmers are also suitable (Lee et al. (1997) Nucleic Acids Res. 25:2816). Exemplary fluorophores of interest are further described in WO 01/42505 and WO 01/86001, incorporated herein by reference in their entirety and for all purposes. Fluorophores can be chosen to absorb and emit in the visible spectrum or outside the visible spectrum, such as in the ultraviolet or infrared ranges.

A fluorescence quencher is a moiety that, when placed very close to an excited fluorophore, causes there to be little or no fluorescence. Suitable quenchers described in the art include, but are not limited to, Black Hole Quenchers, rhodamine, tetramethyl rhodamine, pyrene butyrate, cosine nitrotyrosine, ethidium, fluorescein, Malachite green, Texas Red, and DABCYL and variants thereof, such as DABSYL, DABMI and Methyl Red. Fluorophores can also be used as quenchers, because they tend to quench fluorescence when touching certain other fluorophores. Suitable quenchers can be, for example, either chromophores such as DABCYL or malachite green, or fluorophores that do not fluoresce in the detection range when the detection oligonucleotide segment is in the open conformation. Gold nanoparticles, for example, are also suitable as fluorescent quenchers.

Although the tentacle probes of the present invention can contain nuclease susceptible cleavage sites, they need not. Accordingly, in certain embodiments, the tentacle probes will not comprise a nuclease susceptible cleavage site. In certain embodiments, the tentacle probe or cooperative probe of the present invention will comprise two or more signal altering moieties and there will be no nuclease susceptible cleavage site between the signal altering moieties.

C. Conjugation

A variety of methods are available for attaching the detection and capture probes together. In accordance with certain embodiments of the present invention, the capture probe is directly attached to the detection probe. The detection and capture oligonucleotide segments can be attached together by connecting the 3′ end of the detection oligonucleotide with the 5′ end of the capture oligonucleotide or the 5′ end of the detection oligonucleotide with the 3′ end of the capture oligonucleotide, thus forming a single continuous oligonucleotide. If desired, the capture and detection probes can also be connected by attached via 3′ to 3′ or 5′ to 5′ fashion.

Similarly, when there are more than one target analyte binding regions in a continuous oligonucleotide capture probe, these binding regions can also be attached, for example, via 3′ to 3′, 5′ to 5′, 3′ to 5′, or 5′ to 3′ fashion.

In certain alternative embodiments, the detection and capture probes are attached together via a linker, such as a bifunctional linker. Similarly, the detection and capture probes can also be attached via 3′ to 3′, 5′ to 5′, 3′ to 5′, or 5′ to 3′ fashion. Additionally, the detection and capture probes can also be connected through other positions on the those oligonucleotide probes, as long as the connection does not have deleterious affects the hybridization of the stem, the interaction between the two signal altering moieties, and the binding interactions of these probe with the intended target analyte. For example, a capture probe can be attached to the detection probe through one of the two signal altering moieties.

Suitable bifunctional linkers include, but are not limited to, homobifunctional linkers (e.g., 1,4-phenylene diisothiocyanate) or heterobifunctional linkers. For the present application, a heterobifunctional linker is generally advantageous over a homobifunctional linker in that a heterobifunctional linker avoids the undesired nonspecific crosslinking reactions and aggregation problems normally associated with a homobifunctional conjugation reagent and thus provides substantially higher yields and purer products. Use of a linking reagent for preparation of conjugates with biomolecules, such as proteins, lipids, or oligonucleotides, is well known in the art. Such cross-linking agents are described, for example, in Wong, S. S., Chemistry of Protein Conjugation and Cross-linking, CRC Press, Boca Raton, Fla. (1991), pp. 147-164).

A variety of different coupling chemistries may be employed. For example, a suitable heterobifunctional reagent includes a first reactive group (e.g., N-hydroxysuccinimide) specific for the amino groups of the detection probe and a second reactive group (e.g., maleimide) specific for the thiol groups of the capture probe, or vice verse. Exemplary cross-linking agents of this type include the following: N-sulfosuccinimidyl 4-(N-maleimidomethyl)cyclohexane-1-carboxylate (“Sulfo-SMCC”), N-succinimidyl 3-(2-pyridyldithio)propionate; N-succinimidyl maleimidoacetate; N-succinimidyl 3-maleimidopropionate; N-succinimidyl 4-maleimidobutyrate; N-succinimidyl 6-maleimidocaproate; N-succinimidyl 4-(N-maleimidomethyl)cyclohexane-1-carboxylate; N-succinimidyl 4-(p-maleimidophenyl)butyrate; N-sulfosuccinimidyl 4-(p-maleimidophenyl)-butyrate; N-succinimidyl o-maleimidobenzoate; N-succinimidyl m-maleimidobenzoate; N-sulfosuccinimidyl m-maleimidobenzoate; N-succinimidyl p-maleimidobenzoate; N-succinimidyl 4-maleimido-3-methoxybenzoate; N-succinimidyl 5-maleimido-2-methoxybenzoate; N-succinimidyl 3-maleimido-4-methoxybenzoate; N-succinimidyl 3-maleimido-4-(N,N-dimethyl)aminobenzoate; maleimidoethoxy[p-(N-succinimidylpropionate)phenoxy]ethane; N-succinimidyl-4-[(N-iodoacetyl)amino]benzoate; N-succinimidyl 3-maleimido-4-(N,N-dimethyl)aminobenzoate; maleimidoethoxy[p-(N-succinimidylpropionate)-phenoxy]ethane; N-succinimidyl-4-[(N-iodoacetyl)amino]benzoate; N-sulfosuccinimidyl 4-[(N-iodoacetyl)amino]-benzoate; N-succinimidyliodoacetate; N-succinimidylbromoacetate; N-succinimidyl3-(2-bromo-3-oxobutane-1-sulfonyl)propionate; N-succinimidyl 3-(4-bromo-3-oxobutane-1-sulfonyl)-propionate; N-succinimidyl 2,3-dibromopropionate; N-succinimidyl 4-[(N,N-bis(2-chloroethyl)-amino]phenylbutyrate; p-nitrophenyl 3-(2-bromo-3-oxobutane-1-sulfonyl)propionate; p-nitrophenyl-3-(4-bromo-3-oxobutane-1-sulfonyl)propionate; p-nitrophenyl 6-maleimidocaproate; (2-nitro-4-sulfonic acid-phenyl)-6-maleimidocaproate; p-nitrophenyliodoacetate; p-nitrophenyl-bromoacetate; 2,4-dinitrophenyl-p-(β-nitrovinyl)benzoate; N-3-fluoro-4,6-dinitrophenyl)-cystamine; methyl 3-(4-pyridyldithio)propionimidate HCl; ethyl iodoacetimidate HCl; ethyl bromoacetimidate HCl; ethyl chloroacetimidate HCl; N-(4-azidocarbonyl-3-hydroxyphenyl)-maleimide; 4-maleimidobenzoylchloride; 2-chloro-4-maleimidobenzoyl chloride; 2-acetoxy-4-maleimidobenzoylchloride; 4-chloroacetylphenylmaleimide; 2-bromoethylmaleimide; N-[4-{(2,5-dihydro-2,5-dioxo-3-furanyl)methyl}thiophenyl]-2,5-dihydro-2,5-dioxo-1H-pyrrole-1-hexanamide; epichlorohydrin; 2-(p-nitrophenyl)allyl-4-nitro-3-carboxyphenylsulfide; 2-(p-nitrophenyl)allyltrimethylammonium iodide; α,α-bis[{(p-chlorophenyl)sulfonyl}methyl]-acetophenone; α,α-bis[{(p-chlorophenyl)sulfonyl}methyl]-p-chloroacetophenone; α,α-bis[{(p-chlorophenyl)sulfonyl}methyl]-4-nitroacetophenone; α,α-bis[(p-tolylsulfonyl)methyl]-4-nitroacetophenone; α,α-bis[{(p-chlorophenyl)sulfonyl}methyl]-m-nitroacetophenone; α,α-bis[(p-tolylsulfonyl)methyl]-m-nitroacetophenone; 4-[2,2-bis{(p-tolylsulfonyl)methyl}acetyl]-benzoic acid; N-[4[2,2-2{(p-tolylsulfonyl)methyl}acetyl]benzoyl]-4-iodoaniline; α,α-bis[(p-tolylsulfonyl)methyl]p-aminoacetophenone; N—[{5-(dimethylamino)naphthyl}sulfonyl]α,α-bis[(p-tolylsulfonyl)methyl]-p-aminoacetophenone; and N-[4-{2,2-bis(p-tolylsulfonyl)methyl}-acetyl]benzoyl-1-(p-aminobenzyl)diethylenetriaminepentaacetic acid.

The functional groups for coupling the capture probe to the detection probe can be located at any position on the nucleic acid molecule which permits the conjugation of two nucleic acid molecules directly or indirectly through a linker without any deleterious affects, once attached, on the hybridization of the stem, the molecular energy transfer between the two signal altering moieties, and the binding interactions of these probe with the intended target analyte. Suitable functional groups for use in the present invention include, for example, an amino group (primary, secondary), a carboxylic acid, a carbonyl group, halides, a thiol, a hydroxyl group, a hydrazine, a carbohydrazide, etc. These functional groups are readily introduced into various positions on a nucleic acid molecule, such as on the 3′-or 5′ terminus, via an internal nucleotide or sugar moiety, or a signal altering moiety, using conventional chemical methods known in the art.

For example, a nucleic acid molecule containing a 5′ terminal primary aliphatic amine group is readily prepared by using, in the final coupling step, the reagent Aminolink2, a phosphoramidite coupling reagent having a trifluoroacetyl-protected amino side chain, available from Applied Biosystems, Foster City, Calif. (Smith et al. (1987) Nucleic Acids Res. 15:6181; Sproat et al. (1991) Nucleic Acids Res. 19:3749).

In certain embodiments, the linker for connecting the detection and capture probes can comprise a polymeric molecule. Various non-limiting examples of polymeric linkers include, but are not limited to, polyethylene glycol (“PEG”), polyglycolic acid, polylactic acid, polypeptide, oligosaccharide, polyurethane, polyamide, polysulfonamide, polysulfoxide, polyphosphonate, and block copolymers thereof, including polymers composed of units of multiple subunits linked by charged or uncharged linking groups. Suitable polymeric molecules may also have various lengths. For example, a suitable water soluble bifunctional PEG may have a structure of Formula I:

A—(CH₂—CH₂—O)ₙ—B I

wherein A and B are functional groups as described herein above, n is from about 2 to about 10,000, from about 2 to about 1,000, from about 2 to about 500, from about 2 to about 100, from about 2 to about 50, or from about 2 to about 20.

In general, it is desired that the linker is as short as possible. This is because that the cooperativity of the binding interactions of the detection and capture oligonucleotide probes is significantly demised as the linker length increases.

Exemplary divalent PEGs include, but are not limited to, ethylene glycol, diethylene glycol, triethylene glycol, tetraethylene glycol, pentaethylene glycol, hexaethylene glycol. Other water soluble polyalkylene glycols are also suitable for use in the present invention.

In certain embodiments, the capture and detection probes are indirectly linked together by a solid support. For example, they can be attached to a common surface in close proximity in space to each other so that the target analyte is able to interact with both the first and second target binding region simultaneously and cooperatively. In certain other embodiments, the linkage of the two probes to each other is not by a solid surface but by a more conventional type of linker, such as, for example, a chemical linker, as described herein.

D. Design of Tentacle Probes

In order to design tentacle probes that function optimally under a given set of assay conditions, it is useful to understand how their detectable signal changes (such as the fluorescent intensity) with temperature in the presence and in the absence of their target analytes. In certain embodiments, when the tentacle probes have a fluorophore as their first signal altering moieties and a quencher as their second signal altering moieties, in the absence of a target analyte, tentacle probes exist in a closed conformation at lower temperatures, the fluorophore and the quencher are held in close proximity to each other by a hairpin stem, and there is no fluorescence. However, at high temperatures the helical order of the stem gives way to a random-coil conformation, separating the fluorophore from the quencher and restoring fluorescence. The temperature at which the stem melts depends upon the GC content and the length of the stem sequence.

In certain embodiments, if a target is added to a solution containing a tentacle probe at temperatures below the melting temperature of its stem of the detection probe, the tentacle probe spontaneously binds to its target, dissociating the stem, and turning on its fluorescence.

E. The fluorescence of the probe-target hybrid may also be affected significantly by temperature.

At low temperatures, the probe-target hybrid remains brightly fluorescent, but as the temperature is raised the probe dissociates from the target and tends to return to its hairpin state, diminishing the fluorescence significantly. The temperature at which the probe-target hybrid melts apart also depends upon the GC content and the length of the detection oligonucleotide probe sequence. The longer the probe and the higher its GC content, the higher the melting temperature of the probe-target hybrid. It is important to note that the probe-target hybrid melting temperature can be adjusted independently from the melting temperature of the stem by selecting a target region of appropriate length. In certain embodiments, the tentacle probe is suitable for assays that are performed below 55° C., because below 55° C. the free tentacle probe remains dark, yet the probe-target hybrids form spontaneously and are stable.

The process of tentacle probe design begins with the selection of the sequences for both detection and capture probes, where the capture probe is the single stranded extending away from the hairpin and the detection probe is contained within the hairpin structure. When the tentacle probe is designed to detect the synthesis of products during polymerase chain reactions, any region within the amplicon that is outside the primer binding sites are suitable. The capture probe sequence of the tentacle probe is selected in such a length that at the annealing temperature of the PCR it is able to bind to its target. In order to discriminate between amplicons that differ from one another by as little as a single nucleotide substitution, the length of the capture probe sequence is preferably such that it dissociates from its target at temperatures of about 5 to about 70° C. or about 7 to about 10° C. higher than the annealing temperature of the PCR. When single-nucleotide allele discrimination is not desired, longer and more stable probes can be chosen. The detection and capture probes may have the same or different melting temperatures.

In an exemplary embodiment, the detection probe is located between 1 and 5 nm, or the equivalent distance of about 5 and 15 basepairs, away from the 3′ end of the capture probe. The direction is important in order to insure that the extension of the probe by the polymerase does not occur. The melting temperature of the probe-target hybrid can be predicted using the ‘percent-GC’ rule or ‘nearest neighbor’ rules well known in the art. In general, the prediction is made for the probe sequence alone before choosing the stem sequences. In exemplary embodiments, the lengths of the detection and capture probes each can independently falls in the range between about 5 and about 1,000, about 10 to about 100, about 15 to about 100, about 15 to about 75, about 15 to about 50, about 15 to about 40, or about 15 to about 30 nucleotides.

In certain embodiments, after selecting the probe sequences, two arm sequences, which can form a stem, can be added on either side of the detection probe sequence, or simply one arm which is complementary to the distal end of the detection probe. The length and the GC content of the end sequence can be designed in such a way that at the annealing temperature of the PCR, and in the absence of the target analyte, the detection probe of the tentacle probe remains in the closed conformation and non-fluorescent. This can be ensured by choosing a stem that melts at a temperate at least about 10° C., at least about 15° C., at least about 20° C., or about 15 to about 30° C. higher than the annealing temperature of the PCR. The melting temperature of the stem is affected by both the length and GC content of the stem sequence. The melting temperature of the stem, however, is not well predicted by the percent-GC rule, since the stem is created by intramolecular hybridization. Instead, a DNA folding program, such as the Zuker DNA folding program is well suited for this purpose. In general, 5 basepair-long GC-rich stems melt between 55 and 60° C., 6 basepair-long GC-rich stems melt between 60 and 65° C., 7-basepair long GC-rich stems melt between 65 and 70° C., 8 basepair-long GC-rich stems melt between 70 and 75° C., 9 basepair-long GC-rich stems melt between 75 and 80° C., and 10-basepair long GC-rich stems melt between 80 and 85° C. Although any arbitrary sequence can be used in designing the stems, in certain embodiments, guanosine residues are not used near the end to which the fluorophore is attached to avoid undesirable interactions between the fluorophore and the guanosines. However, guanosine residues may be used near the end where the quencher is attached. For some fluorophores, this may provide some advantages as guanosine residues tend to quench some fluorophores. Longer stems are be used to enhance the specificity of the tentacle probe of the present invention.

Suitable stem sequence lengths for the detection probe include, but are not limited to, from about 4 to about 10, from 10 to about 100, from about 15 to about 50, from about 15 to about 40, or from about 15 to about 30 bases. In some embodiments, the arms or a portion of at least one arm is also a portion of the nucleic acid sequence recognizable by a target analyte. For instance, in some embodiments, some of the stem sequences may overlap with the target analyte binding sequence. As such, these overlapping stem sequence functions both as a complementary end sequence to hybridize with the other end sequence to form a hairpin in the absence of the target analyte and as a part of the target analyte binding sequence to bind the target analyte in the presence of the target.

The detection and capture probe of the tentacle probe may independently have a various secondary structures. In certain embodiments, in the absence of the target analyte, the detection probe of the tentacle probe is in the hairpin structure and does not contain other structures that either do not place the two single altering moieties in the immediate vicinity of each other, or that form longer stems than intended. The former will cause high background signals, and the latter will make the tentacle probes sluggish in binding to target analytes. A folding of the selected sequence by the Zuker DNA folding program can be used to reveal such problems. If unexpected secondary structures result from the choice of the stem sequence, a different stem sequence can be chosen. If, on the other hand, unexpected secondary structures arise from the identity of the target analyte binding sequence, the frame of the target analyte binding sequence can be moved along the target sequence to obtain a target analyte sequence that is not self-complementary. Small stems within the probe's hairpin loop that are 2- to 3-nucleotides long do not adversely affect the performance of the tentacle probes of the present invention.

As with PCR primers, the sequence of the tentacle probe can be compared with the sequences of the primers, using a primer design software program to make sure that there are no regions of substantial complementarity that may cause the tentacle probe to bind to one of the primers, causing primer extension. Also, the primers that are used are designed to produce a relatively short amplicon. In general, the amplicons are preferably less than about 150-basepairs long. When the tentacle probes are used as internal probes, they must compete with the other strand of the amplicon for binding to the strand that contains their target sequence. Having a shorter amplicon allows the tentacle probes to compete more efficiently, and therefore produces stronger detectable signals during real-time PCR. In addition, smaller amplicons result in more efficient amplification. The signal intensity of the tentacle probe can also be increased by performing asymmetric PCR, in which the primer that makes the strand that is complementary to the tentacle probe is present at a slightly higher concentration than the other primer.

### Applications:

The binding interaction of the tentacle probe with a target analyte can be monitored by the detection probe with an interactive label pair (e.g., the first and second signal altering moieties) as a donor-acceptor pair, such as, for example, a fluorophore-quencher pair. The detectable signal can be measured at one or more discrete time points as in an end-point assays or continuously monitored in real-time as in a continuous assay. Detection of the signal can be performed in any appropriate way based, in part, upon the type of reporter or labeling molecule or employed as known in the art. In some embodiments, the signal can be compared against a control signal or standard curve. Non-limiting examples of existing apparatuses that may be used to monitor the reaction in real-time or take one or more single time point measurements include, Models 7300, 7500, and 7700 Real-Time PCR Systems (Applied Biosystems, Foster City, Calif.); the MyCyler and iCycler Thermal Cyclers (Bio-Rad, Hercules, Calif.); the Mx3000P™ and Mx4000® (Stratagene®, La Jolla, Calif); the Chromo 4™ Four-Color Real-Time System (MJ Research, Inc., Reno, Nev.); and the LightCycler® 2.0 Instrument (Roche Applied Science, Indianapolis, Ind.).

The tentacle probe of the present invention can be used in homogenous or heterogeneous assays. When used in a heterogeneous assay, the tentacle probe or a target analyte in a sample can be immobilized onto a solid support. For use herein, the terms solid support and solid surface are used interchangeably. Solid supports are known to those skilled in the art and include the walls of wells of a reaction tray, test tubes, polystyrene beads, magnetic beads, microarrays, porous matrices, microfluidic channels, nitrocellulose strips, membranes, microparticles such as latex particles, sheep (or other animal) red blood cells, duracytes and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional molecule which has the ability to attract and immobilize the capture reagent. The additional molecule can include a charged substance that is oppositely charged with respect to the capture reagent itself or to a charged substance conjugated to the capture reagent. As yet another alternative, the molecule can be any specific binding member which is immobilized upon (attached to) the solid support and which has the ability to immobilize the capture reagent through a specific binding reaction. The molecule enables the indirect binding of the capture reagent to a solid support material before the performance of the assay or during the performance of the assay. The solid phase thus can be, for example, a plastic, derivatized plastic, magnetic or non-magnetic metal, glass or silicon surface of a test tube, microtiter well, sheet, bead, microparticle, chip, sheep (or other suitable animal's) red blood cells, duracytes and other configurations known to those of ordinary skill in the art. The nucleic acids, polynucleotides, primers and probes of the invention can be attached to or immobilized on a solid support individually or in groups of at least 1, 2, 5, 8, 10, 12, 15, 20, or 25 distinct polynucleotides of the invention to a single solid support. In addition, polynucleotides other than those of the invention may be attached to the same solid support as one or more polynucleotides of the invention.

To facilitate its application in solid phase assays, the tentacle probe can also comprise a linkage moiety that is readily captured by a solid support. In general, the linkage moiety has a first end attached to the tentacle probe via the detection probe or the capture probe and a second end to interaction with the solid phase.

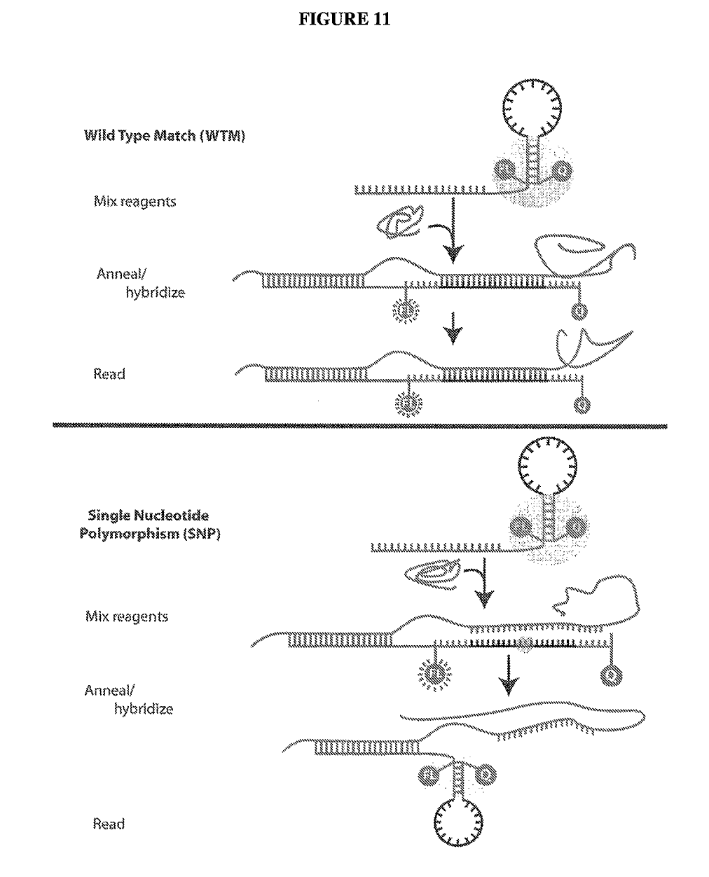

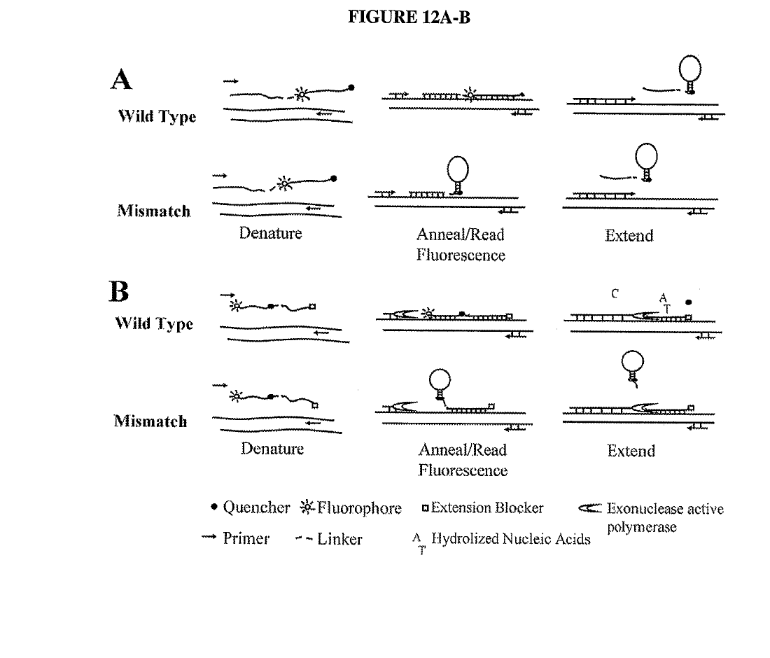

The tentacle probe according to the present invention, can be utilized in detection assays. They can also be used as detectors in amplifications assays, and can be added prior or during amplification, in which case quantitative results as to the initial concentration of amplifiable target may be obtained. Amplification reactions include the polymerase chain reaction (PCR), strand displacement amplification (SDA, e.g., Walker et al., 1992, Proc. Natl. Acad. Sci. U.S.A. 89:392 396)), nucleic acid sequence based amplification (NASBA, Cangene, Mississauga, Ontario; e.g., Compton, 1991, Nature 350:91), transcription mediated amplification (TMA), the ligase chain reaction (LCR; e.g., Wu and Wallace, 1989, Genomics 4:560), rolling circle amplification, self-sustained sequence replication (3 SR; Guatelli et al. 1990, Proc. Natl. Acad. Sci. USA 87, 1874-1878) and RNA-directed RNA amplification catalyzed by an enzyme such as Q-beta replicase (Lizardi et al. 1988, Bio/Technology 6, 1197-1202). Multiple probes for multiple targets may be used in a single reaction tube or other container for multiplex assays. Methods of performing amplification reactions are well known in the art and thus not described herein. The amplification reaction can occur in the presence or absence of exonuclease polymerase. Exemplary methods of using exemplary tentacle probes in amplification reactions are shown in FIGS. 11, 12A, 12B, and 26.

In some embodiments the time that signal is read is important. For example, qPCR fluorescence can be read at the beginning of each cycle or at the end of each cycle or can be monitored continuously. In some embodiments, it is preferable to read the fluorescence following an annealing or hybridization step. In some embodiments, the temperature is adjusted following said hybridization step to a temperature that is of the desired stringency; that is to a temperature where nonspecific analyte does not produce a detectable signal, yet where the target analyte is detectable.

In some embodiments, a prehybridization signal and a post hybridization signal may be desirable in order to determine the presence of a potential change in signal. In other embodiments, standard curves may be desirable in order to compare a generated signal for the purposes of identifying and/or quantifying the presence of an analyte.

Tentacle probes of the present invention can be used for detecting non-multiplied and multiplied target analyte. In certain embodiments of the present invention, when the analyte is multiplied nucleic acid, the capture probe will not function as a primer, e.g., it will bind to a region outside of the primer binding sites and/or it will be non-extendable and/or it will be blocked at the 3′ end to prohibit polymerase catalyzed extension. In certain embodiments of the present invention when the analyte is nucleic acid, including multiplied nucleic acid, the capture probe and detection probe will comprise a sequence that is complementary to the same strand of nucleic acid and to a sequence that is present on the nucleic acid before amplification.

The present invention can be practiced with any known array, including microarrays and biochips and varations thereof. In an exemplary embodiment, a microarray for use in the present invention, comprises a plurality of “spots”, each spot comprising a defined amount of one or more cooperative probe, e.g., tentacle probe, immobilized onto a defined area of a substrate surface for specific binding to an analyte. Methods of making and using arrays are known in the art and not described herein in detail. When using cooperative probes, i.e., tentacle probes, of the present invention in microarray format, several of the steps typically required for analyte detection can be foregone. For example, traditional methods of running microarray experiments require at least the following three steps prior to detection: 1) sample labeling 2) hybridization 3) removal of unbound sample. With exemplary tentacle probes of the present invention, there is no need to label the sample because the signaling moiety is in the tentacle probe. Because unbound sample does not possess a label, there is also no need to remove unbound sample. Accordingly, the present invention provides methods of detecting the target analyte using a tentacle probe array comprising the steps of contacting the sample to the array and measuring changes in signal. In certain exemplary embodiment, the methods do not include a step of labeling the sample and/or removing unbound sample. In certain exemplary embodiments, the signal will be measured in real time.

The present invention also relates to a kit for practicing the various embodiments disclosed herein. The kit can comprise one or more tentacle probes to produce one more target analyte specific signals. The kit can also include the nucleotide specific amplification primers, comprising sequences, including but not limited to, one or more universal sequences and/or code sequences, which in some embodiments provide hybridization targets for the detection polynucleotides. The kit can further comprise a polymerase suitable to amplify a target sequence and/or a polymerase having 5′-3′ nuclease activity. In various embodiments, kits can further comprise moieties suitable for producing a detectable signal or reporter molecules suitable for monitoring, for example, the accumulation of the nucleotide specific target sequence or modification of a detection polynucleotide, as described above.

### Example I

### Examples of a Tentacle Probe

Examples for the tentacle probes of the present invention are shown in the figures. Additional detection probes or capture probes can potentially be added to create higher order tentacle probes. All of these detection probes can potentially be substituted with aptamer technology for use with proteins as well. Additionally, any of these detection probes may be used in solution without the need of connecting to the surface.

### Example II

### Examples of a Cooperative Probe Assay

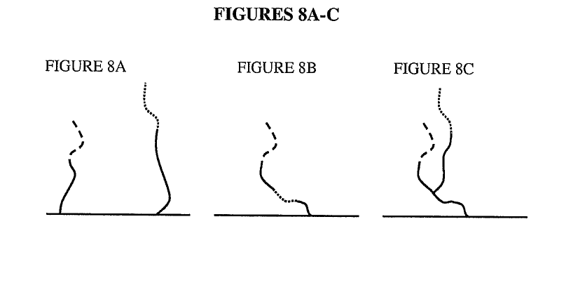

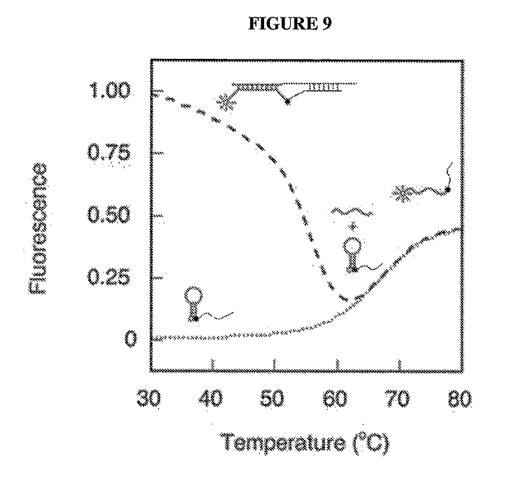

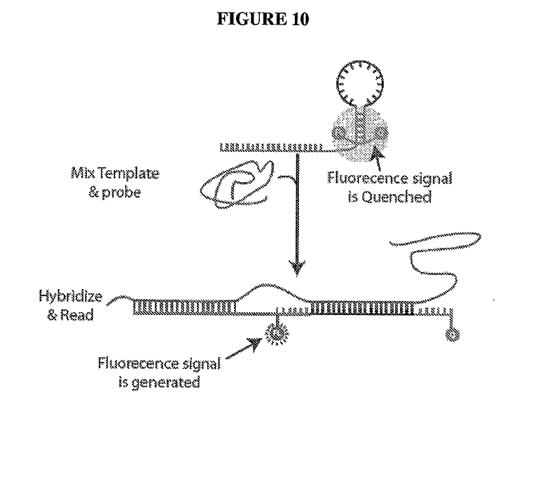

Examples for CPA are shown in FIG. 8. Additional probes can potentially be added to create higher order CPA. All of these probe-based models are also applicable to aptamers, peptides, antibodies or other capture molecules as well. Additionally, any of these embodiments may be used in solution without need for a stem connecting to the surface.

### Example III

### Design of an Exemplary Tentacle Probe for Detection of Anthrax

Genome surrounding anthrax capture probe (underlined) and detection probe in bold is shown below (SEQ ID NO:1):

cgaactcatt gaactaactg ataagagcat gaatacattg attaaaatgt ccagtgtacc
agaaaataga attttagatg gcggaaaagc taatatagta aagtaataat tttatttatg
aatttacttc taaaaagcag atagaaataa aattctagtt ttagacagga gattcgatat

The detection sequence is TGG CGG AAA AGC TAA TAT AGT AA (SEQ ID NO:2) with thermodynamic parameters: dH of −169.8, dS of −0.4878, and Tm of 53.3° C. The capture sequence is GAT TAA AAT GTC CAG TGT ACC AG (SEQ ID NO:3) with thermodynamic parameters: dH of −175.8, dS of −0.5093, and Tm of 51.4° C. All the Tm's were measured in the presence of 50 mM NaCl. Adding 4 mM MgCl₂ increases the Tm by 6-8° C.

In the following oligonucleotide sequences, noncoding sequences are shown in lower cases. The first tentacle probe has a structure of

5′ (GAT TAA AAT GTC CAG TGT ACC AG)-PEG spacer-
quencher (g TGG CGG AAA AGC TAA TAT AGT AA gccac)-
fluorophore 3′

wherein each arm contains five nucleotides in length and the detection probe has thermodynamic parameters: dH of −39.8, dS of −0.1232, Tm of 49.9° C. and dG at 45° C. of −0.6. The sequence “GAT TAA AAT GTC CAG TGT ACC AG” is SEQ ID NO:3 and the sequence “g TGG CGG AAA AGC TAA TAT AGT AA gccac” is SEQ ID NO:4. The arms are “gTGGC” (SEQ ID NO:5) and “gccac” (SEQ ID NO:6).

The second tentacle probe has a structure of

5′ (GAT TAA AAT GTC CAG TGT ACC AG)-PEG spacer-
quencher (g TGG CGG AAA AGC TAA TAT AGT AA
cgccac)-fluorophore 3′

wherein each arm contains six nucleotides in length and the detection probe has thermodynamic parameters: dH of −49.3, dS of −0.148, Tm of 59.9° C. and dG at 45° C. of −2.2. The sequence “GAT TAA AAT GTC CAG TGT ACC AG” is SEQ ID NO:3 and the sequence “g TGG CGG AAA AGC TAA TAT AGT AA cgccac” is SEQ ID NO:7. The arms are “gTGGCG” (SEQ ID NO:8) and “cgccac” (SEQ ID NO:9).

The third tentacle probe has a structure of

5′ (GAT TAA AAT GTC CAG TGT ACC AG)-PEG spacer-
quencher (g TGG CGG AAA AGC TAA TAT AGT AA
ccgccac)-fluorophore 3′

wherein each arm contains seven nucleotides in length and the detection probe has thermodynamic parameters: dH of −59.3, dS of −0.1751, Tm of 65.6° C. and dG at 45° C. of −3.6. The sequence “GAT TAA AAT GTC CAG TGT ACC AG” is SEQ ID NO:3 and the sequence “g TGG CGG AAA AGC TAA TAT AGT AA ccgccac” is SEQ ID NO:10. The arms are “gTGGCGG” (SEQ ID NO:11) and “ccgccac” (SEQ ID NO:12).

The fourth tentacle probe has a structure of

5′ (GAT TAA AAT GTC CAG TGT ACC AG)-PEG spacer-
quencher (gg TGG CGG AAA AGC TAA TAT AGT AA
ccgccacc)-fluorophore 3′

wherein each arm contains eight nucleotides in length and the detection probe has thermodynamic parameters: dH of −67.3, dS of −0.1961, Tm of 70.0° C. and dG at 45° C. of −4.9. The sequence “GAT TAA AAT GTC CAG TGT ACC AG” is SEQ ID NO:3 and the sequence “gg TGG CGG AAA AGC TAA TAT AGT AA ccgccacc” is SEQ ID NO:13. The arms are “ggTGGCGG” (SEQ ID NO:14) and “ccgccacc” (SEQ ID NO:15).

The fifth tentacle probe has a structure of

5′ (GAT TAA AAT GTC CAG TGT ACC AG)-PEG spacer-
quencher (ggg TGG CGG AAA AGC TAA TAT AGT AA
ccgccaccc)-fluorophore 3′

wherein each arm contains nine nucleotides in length and the detection probe has thermodynamic parameters: dH of −75.3, dS of −0.2169, Tm of 74.0° C. and dG at 45° C. of −6.3. The sequence “GAT TAA AAT GTC CAG TGT ACC AG” is SEQ ID NO:3 and the sequence “ggg TGG CGG AAA AGC TAA TAT AGT AA ccgccaccc” is SEQ ID NO:16. The arms are “gggTGGCGG” (SEQ ID NO:17) and “ccgccaccc” (SEQ ID NO:18).

For comparison, the following five conventional beacons are also designed:

5′ quencher
(g TGG CGG AAA AGC TAA TAT AGT AA gccac)-
fluorophore 3′
5′ quencher
(g TGG CGG AAA AGC TAA TAT AGT AA cgccac)-
fluorophore 3′
5′ quencher
(g TGG CGG AAA AGC TAA TAT AGT AA ccgccac)-
fluorophore 3′
5′ quencher
(gg TGG CGG AAA AGC TAA TAT AGT AA ccgccacc)-
fluorophore 3′
5′ quencher
(ggg TGG CGG AAA AGC TAA TAT AGT AA ccgccaccc)-
fluorophore 3′

For real-time PCR, the following two primers were designed: AA CTA ACT GAT AAG AGC AT (SEQ ID NO:19), which has a Tm of 54.6° C. under PCT reaction conditions, and TA TCG AAT CTC CTG TCT (SEQ ID NO:20), which has a Tm of 54.9° C. under PCR reaction conditions.

For characterization of the five tentacle probes, the following four anthrax synthetic nucleotides were employed:

1. Anthrax:

(SEQ ID NO: 21)

gaatacattg attaaaatgt ccagtgtacc agaaaataga
attttagatg gcggaaaagc taatatagta aagtaataat

2. Anthrax with SNP in capture probe:

(SEQ ID NO: 22)

gaatacattg attaaaatat ccagtgtacc agaaaataga
attttagatg gcggaaaagc taatatagta aagtaataat

3. Anthrax with SNP in detection probe:

(SEQ ID NO: 23)

gaatacattg attaaaatgt ccagtgtacc agaaaataga
attttagatg gcggaaaaga taatatagta aagtaataat

4. Anthrax with SNP in both probes:

(SEQ ID NO: 24)

gaatacattg attaaaatat ccagtgtacc agaaaataga
attttagatg gcggaaaaga taatatagta aagtaataat

The actual anthrax and Bt DNA from USAMRIID were also obtained for analysis using real-time PCR.

### Example IV

### Characterization of Exemplary Tentacle Probes

Melting curves of each Tentacle Probes were generated for specific and nonspecific analyte on a Stragene Mx4000 plate reader or comparable product. All probes and target sequences were suspended in TE buffer (10 mM Tris-Cl, 1 mM EDTA, pH 7.0) with 0.18 M NaCl and 0.1% SDS. 20 μL of solutions with final probe concentration of 50 nM and target concentrations of 50 nM, 500 nM, 5 μM and 50 μM were prepared. Fluorescence was monitored from 90° C. to 15° C. at the end of a fifteen minute incubation period following each 1° C. increment. The experiment was also repeated from 15° C. to 90° C. An example of this type of analysis is shown in FIG. 24a& b. Melting peaks are calculated by subtracting the fluorescence at each temperature from the fluorescence at the temperature preceding it. The peak is the highest fluorescent change from one temperature to the next and corresponds to the temperature at which approximately half the bound probes have melted. One potential benefit of cooperativity is that the temperature at which the melting peak occurs for the given experiments shifts less than 10% from the highest value or in ideal cases, less than 5% as shown in FIG. 24a.

### Example V

### Measuring Kinetics of Exemplary Tentacle Probes

Kinetics were measured on a Victor2 plate reader (Perkin Elmer) at room temperature. Due to the rapidity of the TP reactions, low concentrations were used and samples had to be run individually. For the Tentacle Probes, 20 μL of each probe (100 nM) was inserted into the plate. 20 μL of an excess of target (1 μM) was quickly (<5 s) added to each well and the measurements were started. Ninety-nine measurements were taken over ten minutes. The experiment was repeated three times. Molecular beacon kinetics were measured identically except 10 μM target was used and fluorescence was monitored over one hour.

Fluorescent data was plotted against time and the rate constants were fit using the kinetic equation for polynucleotide reactions in an excess of target by minimizing the sum of square errors. The rate constants from each of three trials were averaged and plotted against stem strength with 95% confidence intervals.

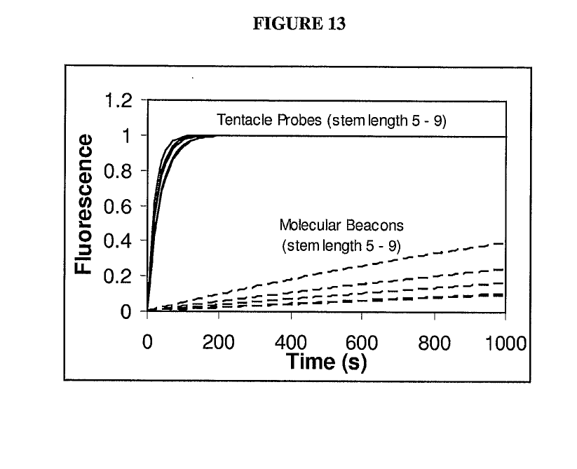

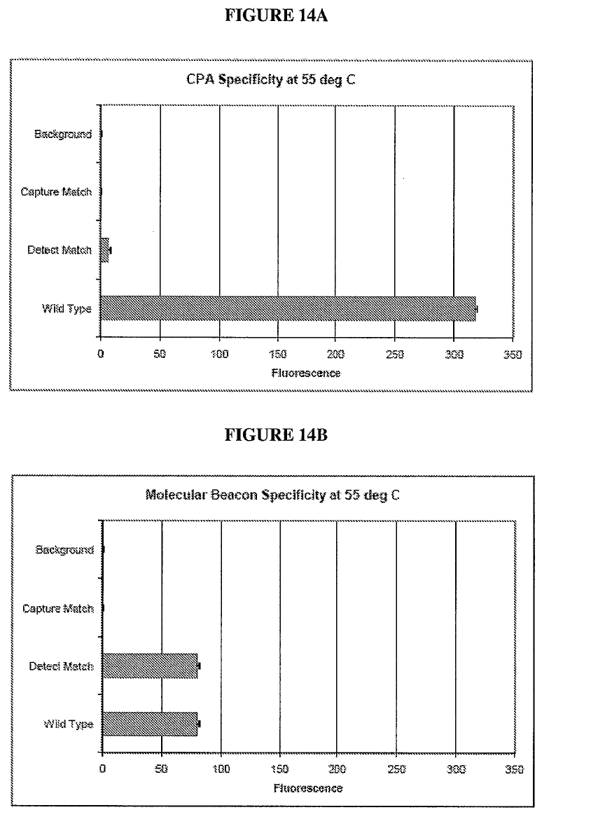

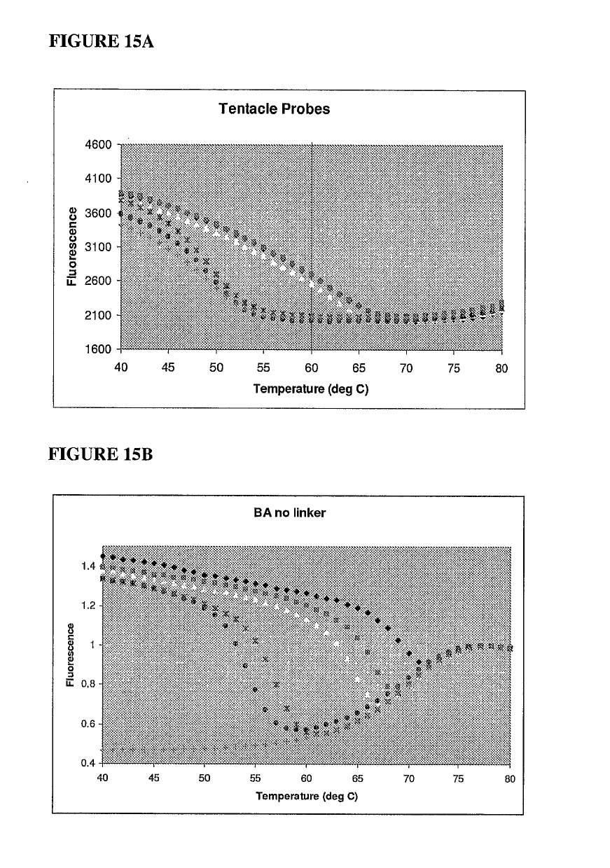

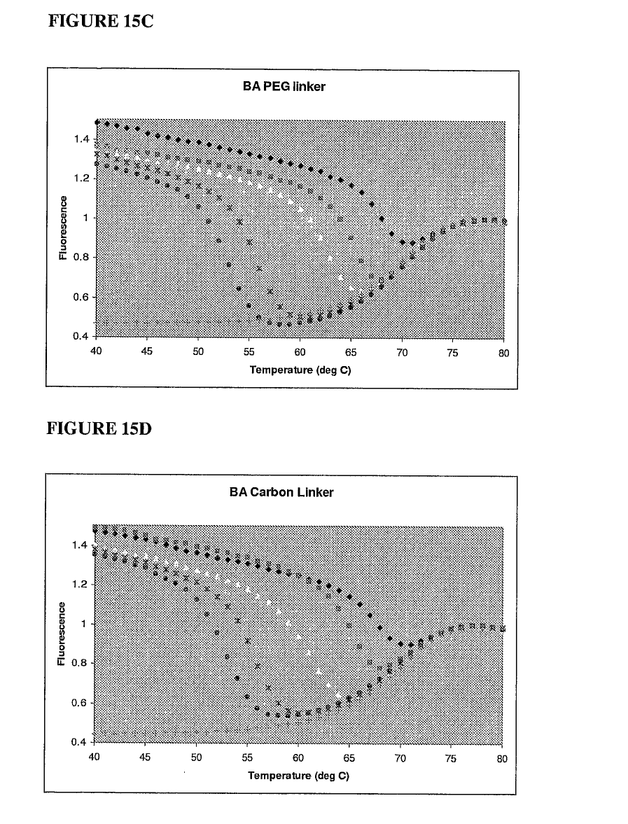

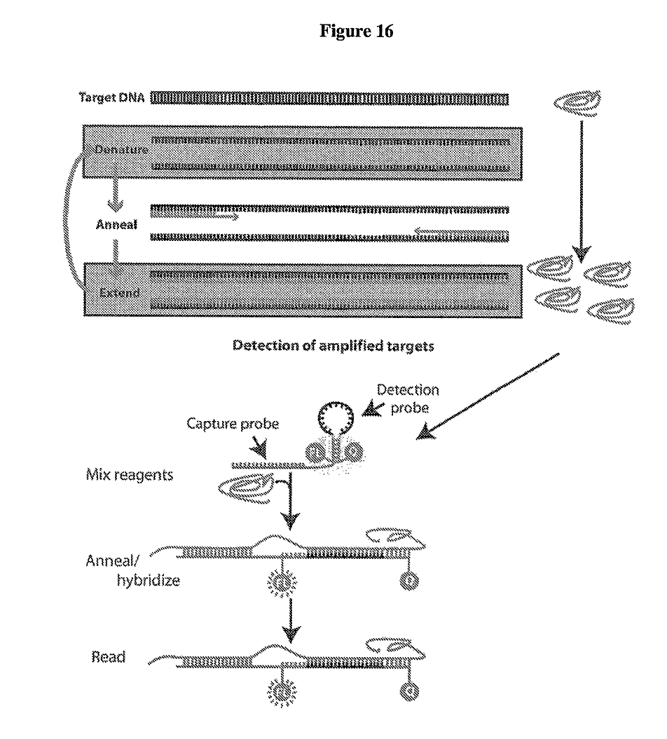

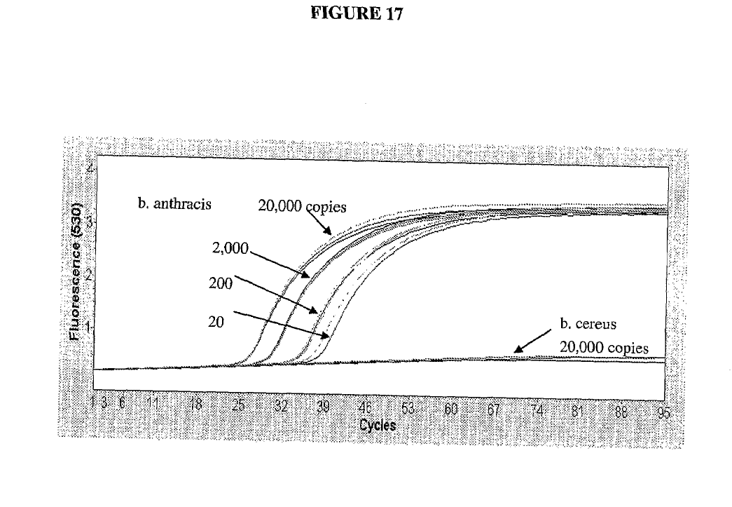

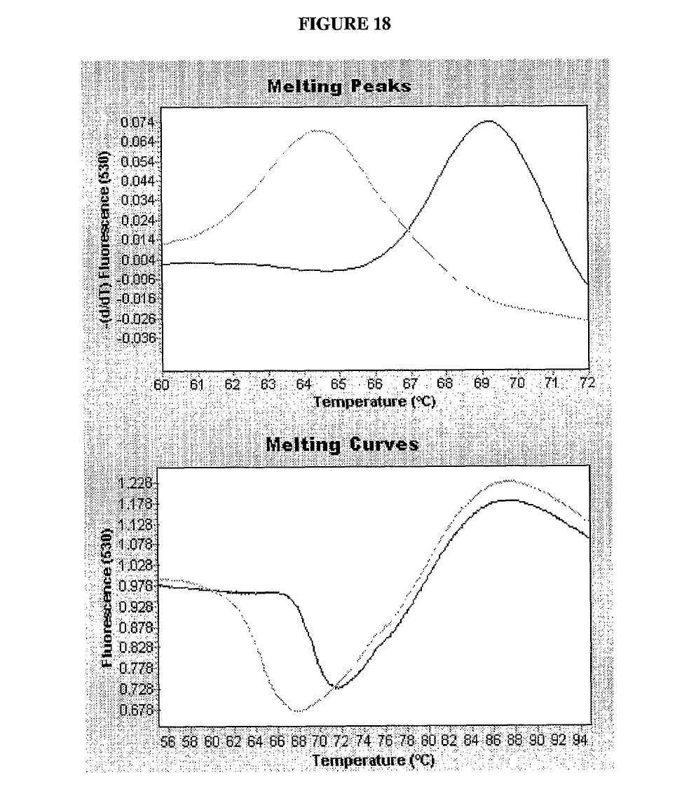

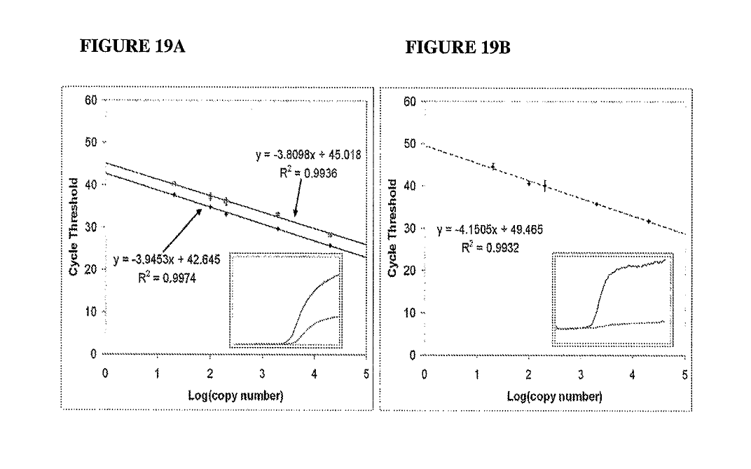

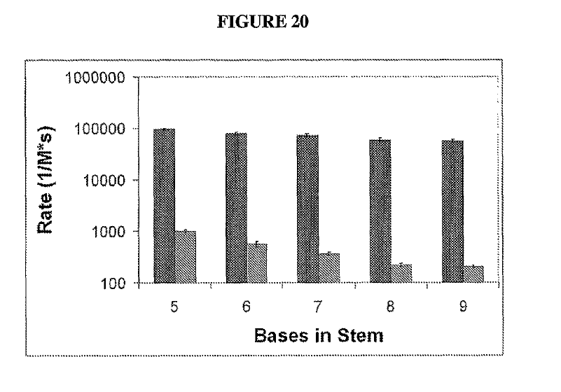

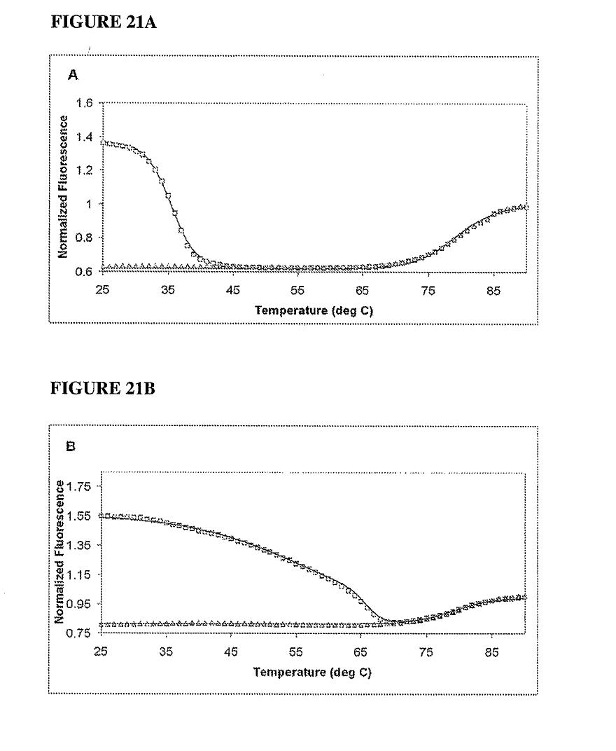

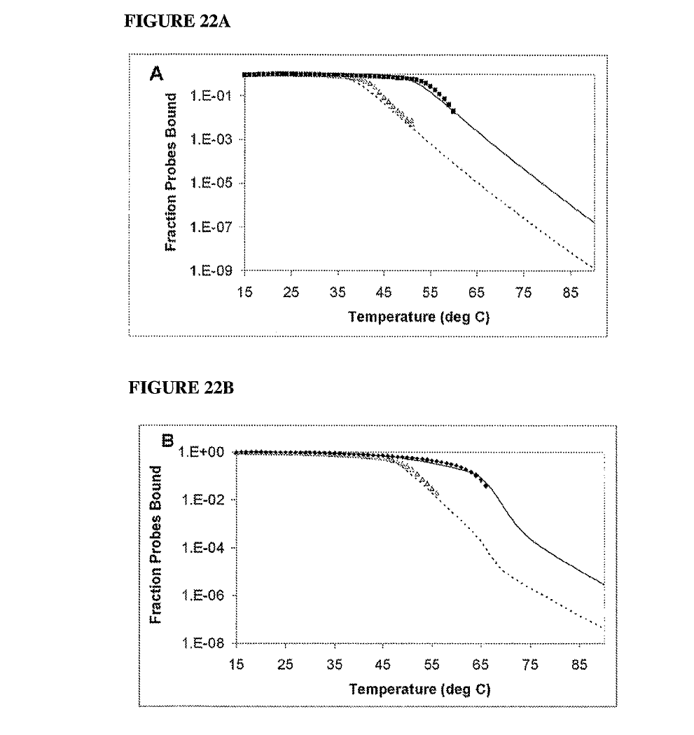

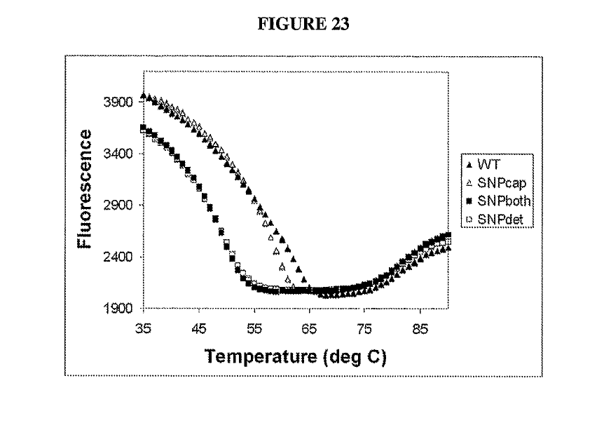

F=Fₘₐₓ(1−e^{(-k}^{_{f}}^{T})t) (1)

Where F is fluorescence, Fₘₐₓ is the maximum fluorescence achieved at equilibrium, k_{f} is the effective forward rate constant, T is the target concentration and t is time. Results are shown in FIGS. 13 & 20.

### Example VI

Measuring thermodynamic parameters of exemplary tentacle probes Fₚₑₙ refers to the collective enthalpic and entropic penalties (Fₚₑₙ=e^{(-ΔHpen/T+ΔSpen)/R}), and all other variables are as previously defined.

Fluorescence as a function of temperature can be adapted from models for molecular beacons (19,20) by:

$$F = α \frac{( {C}_{det} + {C}_{both} )}{{P}_{o}} + β \frac{( {C}_{cap , cl} + {P}_{cl} )}{{P}_{o}} + γ \frac{( {C}_{cap , op} + {P}_{op} )}{{P}_{o}} \qquad ( 8 )$$

Where F is fluorescence as a function of temperature. Alpha, beta and gamma refer to characteristic fluorescence of bound probes, closed probes (subscript cl) and random coil probes (subscript op) respectively.
For Tₒ>>Pₒ, which eliminates calculation of quadratics:

$$F = α \frac{{K}_{det} + {P}_{L} {F}_{pen} {K}_{det} {K}_{cap}}{{K}_{eff}} ( \frac{{T}_{o} {K}_{eff}}{1 + {T}_{o} {K}_{eff}} ) + β \{ 1 - \frac{{K}_{det} + {P}_{L} {F}_{pen} {K}_{det} {K}_{cap}}{{K}_{eff}} ( \frac{{T}_{o} {K}_{eff}}{1 + {T}_{o} {K}_{eff}} ) \} \frac{1}{1 + {K}_{stem}} + γ \{ 1 - \frac{{K}_{det} + {P}_{L} {F}_{pen} {K}_{det} {K}_{cap}}{{K}_{eff}} ( \frac{{T}_{o} {K}_{eff}}{1 + {T}_{o} {K}_{eff}} ) \} \frac{{K}_{stem}}{1 + {K}_{stem}} \qquad ( 9 )$$

Kₛₜₑₘ is fit first by measuring fluorescence as a function of temperature for beacons with no target and minimizing sum of square errors as performed by Bonnet et al and Tsourkas et al (19, 20):

$$F = β \frac{1}{1 + {K}_{stem}} + γ \frac{{K}_{stem}}{1 + {K}_{stem}} \qquad ( 10 )$$

Next K_{det} is fit on probes with no capture probe (e.g., on a molecular beacon):

$$F = α ( \frac{{T}_{o} {K}_{det}}{1 + {T}_{o} {K}_{det}} ) + β \{ 1 - ( \frac{{T}_{o} {K}_{det}}{1 + {T}_{o} {K}_{det}} ) \} \frac{1}{1 + {K}_{stem}} + γ \{ 1 - ( \frac{{T}_{o} {K}_{det}}{1 + {T}_{o} {K}_{det}} ) \} \frac{{K}_{stem}}{1 + {K}_{stem}} \qquad ( 11 )$$

The thermodynamic parameters necessary for calculating K_{cap} were estimated with Mfold (21) for 0.18 M NaCl (about the same sodium concentration as 1×SSC). Using the Mfold estimates provided theoretical curves that diverged at about 61° C. for differing concentrations of target with TP 5, while adjusting the predicted entropy from −0.4989 to −0.494 kcal mol⁻¹ K⁻¹ moved the divergence to about 65° C., forming a more accurate visual fit to the data. Therefore the latter value was used for all remaining tests. Finally, Fₚₑₙ, was fit to the fluorescent curves of three different dilutions of target mixed with Tentacle Probes using the original equation.

The best fit enthalpies and entropies were used to calculate the equilibrium constants which in turn were used to calculate the amount of analyte bound to the detection probe and producing fluorescence as a function of temperature in an excess of target for molecular beacons for specific (subscript s) and nonspecific (subscript ns) analyte:

$$\begin{matrix}\frac{{C}_{s}}{{P}_{0}} = \frac{{K}_{det , s} \cdot {T}_{0}}{1 + {K}_{det , s} \cdot {T}_{0}} & ( 12 ) \\ \frac{{C}_{ns}}{{P}_{0}} = \frac{{K}_{det , ns} \cdot {T}_{0}}{1 + {K}_{det , ns} \cdot {T}_{0}} \\ And for TP \text{:} & ( 13 ) \\ \frac{{C}_{s}}{{P}_{0}} = \frac{( {K}_{det , s} + {P}_{L} {F}_{pen , s} {K}_{det , s} {K}_{cap , s} ) \cdot {T}_{0}}{1 + {K}_{eff , s} \cdot {T}_{0}} & ( 14 ) \\ \frac{{C}_{ns}}{{P}_{0}} = \frac{( {K}_{det , ns} + {P}_{L} {F}_{pen , ns} {K}_{det , ns} {K}_{cap , ns} ) \cdot {T}_{0}}{1 + {K}_{eff , ns} \cdot {T}_{0}} & ( 15 )\end{matrix}$$

These equations were then matched with normalized fluorescent data in order to verify accuracy of thermodynamic parameters in predictions at the lowest detectable binding levels and to identify trends in binding below the level of detection. Thermodynamic values provided from best fits often fit high level binding tightly at the expense of fitting low level binding data due to inequalities arising from the sum of square errors. A manual adjustment to the best fit (e.g. ±0.6 kcal mol⁻¹ for enthalpy and ±0.0016 kcal mol⁻¹ K⁻¹ for entropy) provided a fit that more thoroughly represented both high and low binding data. Once the parameters provided a perfect visual fit to low level binding as well as high level binding (FIG. 22), they were kept constant in order to make predictions as seen in FIG. 25.

### Example VII

### Testing Specificity of Exemplary Tentacle Probes

The Stratagene Mx4000 plate reader was used to read the fluorescence of 1 μM 9-base stem TP and 1 μM 5-base stem MB in WT and SNP_{det} targets at concentrations of 0, 2 nM, 10 nM, 20 nM, 100 nM, 1 μM, and 10 μM. Higher concentrations of 100 μM and 1 mM SNP_{det} were also used where detection limits had not yet been established. Fluorescence was read at equilibrium for both probe types. The reading was performed at 60° C. for the TP and 55° C. for the MB. Three replicates of each type were performed.

The predicted binding curves as a function of target concentration were generated using best fit thermodynamic parameters for molecular beacons:

$$\begin{matrix}\frac{{C}_{det}}{{P}_{0}} = \frac{( {P}_{0} + {T}_{0} + \frac{1}{{K}_{det}} ) - \sqrt{{( {P}_{0} + {T}_{0} + \frac{1}{{K}_{det}} )}^{2} - 4 {P}_{0} {T}_{0}}}{2 {P}_{0}} \\ And for TP \text{:} & ( 16 ) \\ \frac{{C}_{det} + {C}_{both}}{{P}_{0}} = [ \frac{( {P}_{0} + {T}_{0} + \frac{1}{{K}_{eff}} ) - \sqrt{{( {P}_{0} + {T}_{0} + \frac{1}{{K}_{eff}} )}^{2} - 4 {P}_{0} {T}_{0}}}{2 {P}_{0}} ] \frac{{K}_{det} + {P}_{L} {F}_{pen} {K}_{det} {K}_{cap}}{{K}_{eff}} & ( 17 )\end{matrix}$$

These predictions were compared to experimental data plotted with 95% confidence intervals. Before plotting, data was normalized by subtracting the background fluorescence measured at 0 nM target concentration and dividing by the maximum intensity experienced at 100 μM WT target at room temperature. The background fluorescence level plotted was the average plus one standard deviation of the signals of experiments run below and including the highest concentration that could not be statistically confirmed as having fluorescence greater than the preceding concentrations (t-test, p>0.05). The results together with model predictions are shown in FIG. 25.

### Example VIII

### Exemplary Tentacle Probes for Detection of Baccillus anthracis gyrA Gene

The following tentacle probe was designed for the detection of the Baccillus anhtracis gyrA gene. Fam is Fam fluorescent dye, BHQ is black hole quencher, PEG9 is polyethylene glycol nine carbon chain, and C3 is a 3 carbon blocking group.

(FAM)-CTTCTACGCATGACCATATTC gcgtagaag-(BHQ)-
(PEG9)-ATAAAGGGAAAGTATACCG-C3

The capture probe comprises the sequence ATAAAGGGAAAGTATACCG (SEQ ID NO:25) and the detection probe comprises the sequence CTTCTACGCATGACCATATTC gcgtagaag (SEQ ID NO:26). The arms are represented by CTTCTACGC (SEQ ID NO:27) and gcgtagaag (SEQ ID NO:28). The target binding region comprises the sequence CTTCTACGCATGACCATATTC (SEQ ID NO:37).
Primers that can be used to amplify the target analyte include, for example,

Forward primer:

(SEQ ID NO: 29)

BAGYRA1614F [5′-GGG AAC AAA TGA TGA TGA TTT CGT-
3′]
Reverse primer:

(SEQ ID NO: 30)

BAGYRA1732R [5′-ACT CTG GGA TTT CAT ATC CTT TCG T-
3′]

### Example X

Exemplary Tentacle Probes for Detection of Yersinia pestis Gene

The following tentacle probe was designed for the detection of the Yersinia pestis gene. Fam is Fam fluorescent dye, BHQ is black hole quencher, PEG9 is polyethylene glycol nine carbon chain. T is a thymine base derivatized with FAM.

GAG TAT TCG TCT GGG GG peg9 T (FAM) ccc CGA GGT
TCA GGT GAG CAC Gct cgg gga (BHQ)

The capture probe comprises the sequence GAG TAT TCG TCT GGG GG (SEQ ID NO:31) and the detection probe comprises the sequence ccc CGA GGT TCA GGT GAG CAC Gct cgg gga (SEQ ID NO:32). The arms are represented by ccc CGA G (SEQ ID NO:33) and ct cgggg (SEQ ID NO:34). The target binding region comprises the sequence CGA GGT TCA GGT GAG CAC G (SEQ ID NO:38)
Primers that can be used to amplify the target analyte include, for example,

(SEQ ID NO: 35)

Forward primer: [5′-gcaggaaatgcgcaatgc-3′]

(SEQ ID NO: 36)

Reverse primer: [5′-gggcggatccccacttta-3′]

### Example X

### Discrimination of Difficult Single Nucleotide Polymorphisms

The rate of false positives generated by TaqMan-MGB and Tentacle probes in a qPCR format was compared. The previously developed and optimized TaqMan-MGB assay was compared to a single iteration design of a Tentacle Probe assay for detecting the gyrA gene of B. anthracis and the yp48 gene of Y. pestis. Both of these targets are chromosomal genes that are believed to have significant importance in the ability to detect the presence of these CDC Category A pathogens.

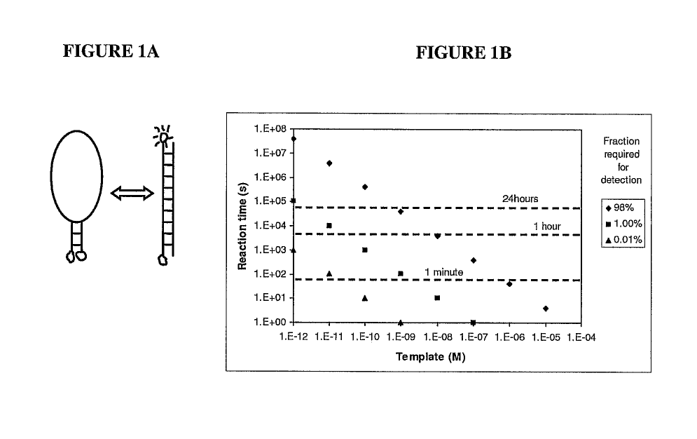

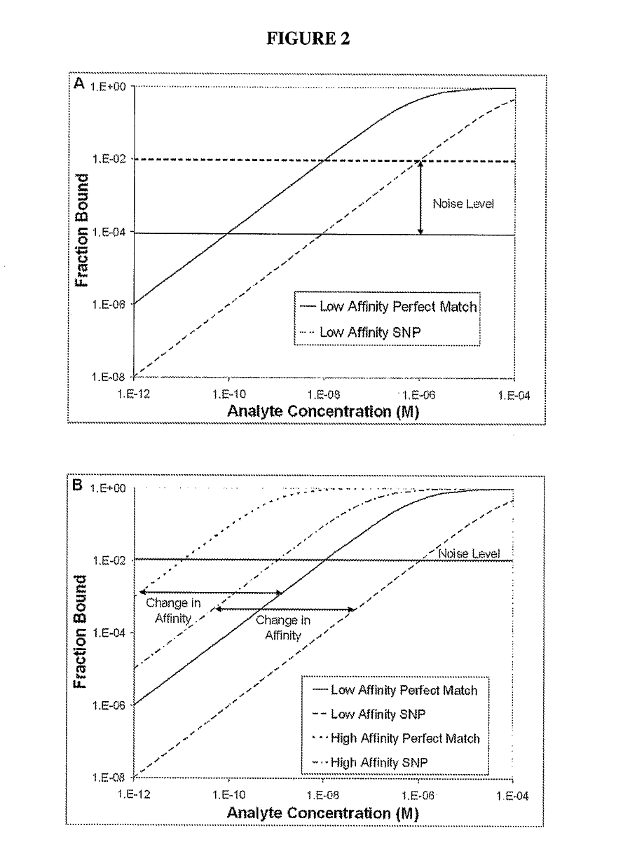

The real-time PCR conditions and probes developed by Chase et al (Clin Chem, 51, (2005) 1778-1785) for yp48 discrimination were used and directly compared with Tentacle Probes targeting the same amplicon. The reaction conditions were 15 μL of master mix (10.2 μL di water, 2 μl 10× reaction buffer in 50 mM MgCl₂, 2 μL of 10× dNTPs, 0.2 μL of forward primer, 0.2 μL of reverse primer, 0.2 μL of 10 μM probe, 0.2 μL of Taq polymerase) with 5 μL of template. TaqMan-MGB assay used Taq platinum polymerase with a 2-min denaturation at 95° C., followed by 95 cycles of 95° C. for 0 seconds and 60° C. for 20 seconds. The Tentacle Probe assay used Taq TSP polymerase (exonuclease deficient) with a 2-min denaturation at 95° C., followed by 95 cycles of 95° C. for 0 seconds, 60° C. for 10 seconds and 70° C. for 10 seconds (mechanism in FIG. 1). Taq TSP polymerase, which is exonuclease deficient, was used for Tentacle Probes to allow fluorescence monitoring at temperatures other than the annealing temperature, such as during the extension step. TaqMan-MGB probes required degradation, so Taq platinum polymerase was used in reactions with TaqMan-MGB probes. Because TaqMan-MGB probes were degraded, fluorescence monitoring at temperatures other than the annealing temperature was not beneficial. Standard dilutions were used from 20 copies to 20,000 copies with 3 replicates each for subsequent round of PCR. Amplification products were run on a gel to verify successful PCR conditions.

The real-time PCR probes developed by Hurtle et al (J Clin Microbiol, 42, (2004) 179-185) for gyrA discrimination were used and directly compared with Tentacle Probes targeting the same amplicon under identical PCR conditions. Those conditions were 15 μL of master mix (10.2 μL di water, 2 μL of 10× reaction buffer in 50 mM MgCl₂, 2 μL of 10× dNTPs, 0.2 μL of forward primer, 0.2 μL of reverse primer, 0.2 μL of 10 μM probe, 0.2 μL of Platinum Taq polymerase) with 5 μL of template. The temperature cycles included a 2-min denaturation at 95° C., followed by 95 cycles of 95° C. for 0 seconds and 60° C. for 20 seconds. Standard dilutions were used from 20 copies to 20,000 copies with 3 replicates each.

Both assays were assessed for specificity with boil preps of 29 environmental samples. The average nucleic acid concentration of each boil prep was 500 ng/μL, and 5 μL was used in each reaction. The reaction conditions for both Tentacle Probes and TaqMan-MGB were 15 μL of master mix (10.2 μL di water, 2 μL 10× reaction buffer in 50 mM MgCl₂, 2 μL of 10× dNTPs, 0.2 μL of forward primer, 0.2 μL of reverse primer, 0.2 μL of 10 uM probe, 0.2 μL of Taq Platinum polymerase) with 5 μL of template undergoing a 2-min denaturation at 95° C., followed by 95 cycles of 95° C. for 0 seconds and 60° C. for 20 seconds. A second set of experiments was performed with an annealing temperature of 67° C. One positive control at 20,000 copies was run simultaneously with 29 boil preps from environmental samples known to contain near neighbors to B. anthracis. Amplification products were run on a gel to verify successful PCR conditions.

The Y. pestis TaqMan-MGB exhibited results similar to those described previously. At all concentrations of near-neighbor Y. pseudotuberculosis, false positives occurred approximately three cycles later than detection of an equivalent concentration of Y. pestis. In contrast, Tentacle Probes had no false positives at any concentration tested. Clean bands approximately 100 bases in size appeared for each PCR product when run in an agarose gel, indicating that lack of amplification was not the cause of the increased specificity.

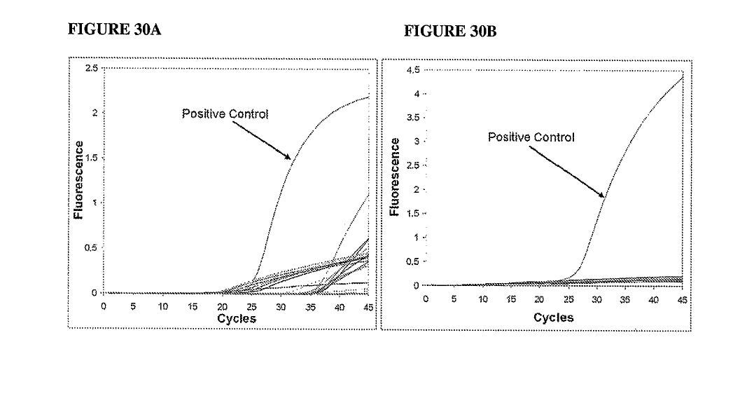

The gyrA assay from 20 to 20,000 purified copies of both wild type and variant produced no false negatives or false positives for both TaqMan-MGB and Tentacle Probes. However, when boil preps of 29 environmental samples were used, TaqMan-MGB had 21 false positives. In contrast, Tentacle Probes had no false positives for any of the samples (FIG. 30). Gels were run of all the PCR products from the boil preps for both TaqMan-MGB and Tentacle Probes. The presence of amplification products in each indicates that failure to amplify was not the cause of the increased specificity.

When Hurtle et al failed to achieve specificity of reaction with a 60° C. annealing temperature even after 6 designs of TaqMan-MGB, they used the best probe design at an elevated annealing temperature, 67° C. (10). Accordingly, the gyrA experiment was repeated with 29 boil preps of environmental samples at this high annealing temperature and still received 7 false positives out of the 29 samples.

The examples set forth above are provided to give those of ordinary skill in the art with a complete disclosure and description of how to make and use the preferred embodiments of the present invention, and are not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A tentacle probe for detecting the presence or absence of a target analyte in a sample, comprising:
a detection probe comprising a first target binding region, wherein said detection probe is in an open conformation when bound to said target analyte and is in a closed conformation when not bound to said target analyte and wherein a change in conformation generates a change in detectable signal; and
a capture probe comprising a second target binding region for said target analyte that is different than the first target binding region; wherein the capture probe is attached to the detection probe and does not function as a primer.

2. (canceled)

3. (canceled)

4. The tentacle probe of claim 1 wherein said detection probe comprises a first arm region and a second arm region that form a stem duplex when the probe is in a closed conformation and are separated when the probe is in an open conformation.

5. (canceled)

6. The tentacle probe of claim 4 wherein said first arm region is attached to a first signal altering moiety and said second arm region is attached to a second signal altering moiety.

7. The tentacle probe of claim 6 wherein the first and second signal altering moieties are part of an energy transfer pair.

8. (canceled)

9. The tentacle probe of claim 4 wherein said first target binding region is physically intermediate to said first and second arm region.

10. The tentacle probe of claim 4 wherein said first or second arm region comprise at least a part of said first target binding region.

11. The tentacle probe of claim 1 wherein the capture probe is attached to the detection probe at its 5′ end.

12. The tentacle probe of claim 1 wherein the capture probe is attached to the detection probe at its 3′ end.

13. (canceled)

14. The tentacle probe of claim 1 wherein said capture probe has no secondary structure.

15. The tentacle probes of claim 1 wherein said capture probe is in an open conformation when bound to said target analyte and is in a closed conformation when not bound to said target analyte and wherein a change in conformation generates a change in detectable signal.

16. The tentacle probe of claim 1 wherein said target analyte is nucleic acid.

17. The tentacle probe of claim 16 wherein the capture probe and the detection probe comprise a sequence that is complementary to the same strand of nucleic acid and the sequence is present on the nucleic acid before amplification.

18. (canceled)

19. (canceled)

20. The tentacle probe of claim 1 wherein the target analyte is a protein, small organic molecule, or cell and the first and second target binding regions comprise aptamers.

21. The tentacle probe of claim 1 wherein the capture probe is directly attached to the detection probe.

22. The tentacle probe of claim 1 wherein the capture probe is attached to the detection probe by a linker.

23. (canceled)

24. (canceled)

25. (canceled)

26. (canceled)

27. (canceled)

28. (canceled)

29. The tentacle probe of claim 1, wherein the 3′ end of the capture probe is a phosphate group.

30. The tentacle probe of claim 1, wherein the 3′ end of the capture probe is a functional group other than hydroxyl (—OH).

31. The tentacle probe of claim 1 wherein the tentacle probe is immobilized on a solid surface.

32. (canceled)

33. A method for detecting the presence or absence of a target analyte in a test sample comprising:
contacting a tentacle probe of claim 1 with the test sample; and
measuring the signal.

34. The method of claim 33 further comprising contacting a second tentacle probe of claim 1 comprising a first and second target binding region specific for a second target analyte and cable of generating a distinguishable signal from said first tentacle probe with the test sample, measuring the signal, and detecting a plurality of different target analytes in the test sample.

35. The method of claim 33 wherein the target analyte is nucleic acid and the method further comprises amplifying the target analyte in the presence of the tentacle probe.

36. (canceled)

37. (canceled)

38. (canceled)

39. (canceled)

40. The method of claim 33 wherein the the presence or absence of a non-multiplied target analyte in a test sample is detected.

41. (canceled)

42. A tentacle probe for detecting a target analyte in a sample, comprising:
a detection probe comprising a first arm region and a second arm region wherein said first arm region and second arm region form a stem duplex when in a closed conformation and wherein the first arm of the first detection probe is attached to a first signal altering moiety, said detection probe comprising a first target binding region; and
a capture probe comprising a third arm region and a fourth arm region wherein said third arm region and fourth arm region form a stem duplex when in a closed conformation and wherein the third arm is attached to a second signal altering moiety, said capture probe comprising a second target binding region, wherein said capture probe is attached to the detection probe and wherein said detection probe and capture probe are in an open conformation when bound to said target analyte and in a closed conformation when not bound to said target analyte and wherein a change in conformation generates a change in detectable signal.

43. A method for detecting the presence or absence of a target analyte in a test sample comprising:
contacting the tentacle probe of claim 42 with the test sample; and
measuring the signal.

44. A tentacle probe for detecting a target nucleic acid in a sample while inhibiting detection of a variant of said target nucleic acid comprising an insertion sequence, comprising
a detection probe comprising a first binding region, wherein said detection probe is in an open conformation when bound to said target analyte and is in a closed conformation when not bound to said target analyte and wherein a change in conformation generates a change in detectable signal; and
a capture probe comprising a second binding region that is different than the first binding region, wherein the capture probe is attached to the detection probe, and wherein the first binding region comprises a sequence that is complementary to a sequence present in the target nucleic acid but not present in the variant, and wherein the second binding region comprises a sequence that is complementary to the insertion sequence of the variant.

45. The tentacle probe of claim 44 wherein said detection probe comprises a first arm region and a second arm region that form a stem duplex when the probe is in a closed conformation and are separated when the probe is in an open conformation.

46. The tentacle probe of claim 45 wherein at least one of said arm regions is attached to a signal altering moiety.

47. The tentacle probe of claim 45 wherein said first arm region is attached to a first signal altering moiety and said second arm region is attached to a second signal altering moiety.

48. The tentacle probe of claim 44 wherein the capture probe is indirectly attached to the detection probe.

49. A method of detecting the presence or absence of a target analyte while inhibiting non-specific detection of a variant of said target analyte comprising an insertion sequence, comprising contacting the tentacle probe of claim 44 with the test sample, and measuring the signal.

50. The method of claim 49 further comprising amplifying the target analyte in the presence of the tentacle probe and further inhibiting amplification of the variant.

51. (canceled)

52. (canceled)

53. A tentacle probe for detecting a target analyte in a sample while inhibiting detection of a variant of said target analyte comprising an insertion sequence, comprising
a detection probe comprising a first target binding region, wherein said detection probe is in an open conformation when bound to said target analyte and is in a closed conformation when not bound to said target analyte and wherein a change in conformation generates a change in detectable signal; and
a capture probe comprising a second target binding region that is different than the first target binding region, wherein the capture probe is attached to the detection probe through a linker and wherein the linker comprises a nucleic acid sequence complementary to the insertion sequence.

54. The tentacle probe of claim 53 wherein said detection probe comprises a first arm region and a second arm region that form a stem duplex when the probe is in a closed conformation and are separated when the probe is in an open conformation.

55. The tentacle probe of claim 54 wherein at least one of said arm regions is attached to a signal altering moiety.

56. A method of detecting the presence or absence of a target analyte while inhibiting non-specific detection of a variant of said target analyte comprising an insertion sequence, comprising
contacting a tentacle probe of claim 53 with the test sample, and
measuring the signal.

57. The method of claim 56 wherein the method further comprises the step of amplifying the target analyte in the presence of the tentacle probe and the method further inhibits amplification of the variant.

58. (canceled)

59. (canceled)

60. A kit for detecting the presence or absence of an analyte in a sample comprising the tentacle probe of claim 1 1 and instructions for using the tentacle probe to detect the presence or absence of the analyte in the sample.

61. A cooperative probe for detecting the presence or absence of an analyte comprising a probe set of two or more attached probes specific for different regions of said analyte, wherein said cooperative probe has at least one of the following:
(i) an observed melting peak temperature that varies no more than about 10% with increasing concentration of the analyte when the concentration of analyte is greater than the concentration of the cooperative probe; and
(ii) at least one of the attached probes will not detectably bind to the analyte without the analyte binding to at least one of the other attached probes.

62. (canceled)

63. (canceled)

64. (canceled)

65. A method for detecting the presence or absence of a target analyte in a test sample comprising contacting a cooperative probe of claim 60 with the test sample; and measuring the signal.

66. A cooperative probe for detecting the presence or absence of an analyte while inhibiting non-specific detection of a variant of said analyte comprising an insertion sequence comprising a probe set of two or more attached probes wherein at least one of said probes is specific for said analyte and one of said probes is specific for said variant and has an observed melting peak temperature that varies no more than about 10% with increasing concentration of the variant analyte when the concentration of the variant analyte is greater than the concentration of the cooperative probe.

67. A method of detecting the presence or absence of a target analyte while inhibiting non-specific detection of a variant of said target analyte comprising an insertion sequence, comprising contacting the tentacle probe of claim 66 with the test sample, and measuring the signal.

68. A kit for detecting the presence or absence of an analyte in a sample comprising the tentacle probe of claim 42 and instructions for using the tentacle probe to detect the presence or absence of the analyte in the sample.

69. A kit for detecting the presence or absence of an analyte in a sample comprising the tentacle probe of claim 44 and instructions for using the tentacle probe to detect the presence or absence of the analyte in the sample.

70. A kit for detecting the presence or absence of an analyte in a sample comprising the tentacle probe of claim 53 and instructions for using the tentacle probe to detect the presence or absence of the analyte in the sample.
